(19) United States
(12) Patent Application Publication
(10) Pub. No.: US 2023/0355625 A1
(43) Pub. Date: Nov. 9, 2023

(54) METHODS OF TREATMENT

(71) Applicant: INTRA-CELLULAR THERAPIES, INC., New York, NY (US)

(72) Inventors: Kimberly VANOVER, New York, NY (US); Robert DAVIS, San Diego, CA (US); Peng LI, New Milford, NJ (US)

(21) Appl. No.: 17/780,660

(22) PCT Filed: Nov. 25, 2020
(86) PCT No.: PCT/US20/62400

§ 371 Date: May 27, 2022

Related U.S. Application Data
(60) Provisional application No. 62/941,542, filed on Nov. 27, 2019.

(51) Int. Cl. A61K 31/519; A61K 45/06; A61P 25/28

(52) U.S. Cl. CPC A61K 31/519; A61K 45/06; A61P 25/28

(57) ABSTRACT

The present disclosure relates to inhibitors of phosphodiesterase 1 (PDE1) useful for the engagement with the central nervous system. The disclosure further relates to the administration of inhibitors of phosphodiesterase 1 (PDE1) for the treatment of a condition, disease or disorder associated with the dopamine D1 receptor intracellular pathway, e.g., a condition, disease or disorder characterized by a deficit in attention, cognition, memory and/or inhibitory processing.

### CROSS-REFERENCE TO RELATED APPLICATIONS

This application claims priority to and the benefit of U.S. Provisional Application Ser. No. 62/941,542, which was filed on Nov. 27, 2019, the contents of which are hereby incorporated by reference in its entirety.

### FIELD OF DISCLOSURE

The field relates to inhibitors of phosphodiesterase 1 (PDE1) useful for the engagement with the central nervous system. The field further relates to the administration of inhibitors of phosphodiesterase 1 (PDE1) for the treatment of a condition, disease or disorder associated with the dopamine D1 receptor intracellular pathway, e.g., a condition, disease or disorder characterized by a deficit in attention, cognition, memory and/or inhibitory processing.

### BACKGROUND OF THE DISCLOSURE

Eleven families of phosphodiesterases (PDEs) have been identified but only PDEs in Family I, the Ca2+-calmodulin-dependent phosphodiesterases (CaM-PDEs), have been shown to mediate the calcium and cyclic nucleotide (e.g. cAMP and cGMP) signaling pathways. The three known CaM-PDE genes, PDE1A, PDE1B, and PDE1C, are all expressed in central nervous system tissue. PDE1A is expressed throughout the brain with higher levels of expression in the CA1 to CA3 layers of the hippocampus and cerebellum and at a low level in the striatum. PDE1A is also expressed in the lung and heart. PDE1B is predominately expressed in the striatum, dentate gyrus, olfactory tract and cerebellum, and its expression correlates with brain regions having high levels of dopaminergic innervation. Although PDE1B is primarily expressed in the central nervous system, it may be detected in the heart. PDE1C is primarily expressed in olfactory epithelium, cerebellar granule cells, and striatum. PDE1C is also expressed in the heart and vascular smooth muscle.

Cyclic nucleotide phosphodiesterases decrease intracellular cAMP and cGMP signaling by hydrolyzing these cyclic nucleotides to their respective inactive 5′-monophosphates (5′AMP and 5′GMP). CaM-PDEs play a critical role in mediating signal transduction in brain cells, particularly within an area of the brain known as the basal ganglia or striatum. For example, NMDA-type glutamate receptor activation and/or dopamine D2 receptor activation result in increased intracellular calcium concentrations, leading to activation of effectors such as calmodulin-dependent kinase II (CaMKII) and calcineurin and to activation of CaM-PDEs, resulting in reduced cAMP and cGMP. Dopamine D1 receptor activation, on the other hand, leads to activation of calcium dependent nucleotide cyclases, resulting in increased cAMP and cGMP. These cyclic nucleotides in turn activate protein kinase A (PKA; cAMP-dependent protein kinase) and/or protein kinase G (PKG; cAMP-dependent protein kinase) that phosphorylate downstream signal transduction pathway elements such as DARPP-32 (dopamine and cAMP-regulated phosphoprotein) and cAMP responsive element binding protein (CREB).

CaM-PDEs can therefore affect dopamine-regulated and other intracellular signaling pathways in the basal ganglia (striatum), including but not limited to nitric oxide, noradrenergic, neurotensin, CCK, VIP, serotonin, glutamate (e.g., NMDA receptor, AMPA receptor), GABA, acetylcholine, adenosine (e.g., A2A receptor), cannabinoid receptor, natriuretic peptide (e.g., ANP, BNP, CNP) and endorphin intracellular signaling pathways.

Phosphodiesterase (PDE) activity, in particular, phosphodiesterase 1 (PDE1) activity, functions in brain tissue as a regulator of locomotor activity and learning and memory. PDE1 is a therapeutic target for regulation of intracellular signaling pathways, preferably in the nervous system, including but not limited to a dopamine D1 receptor, dopamine D2 receptor, nitric oxide, noradrenergic, neurotensin, CCK, VIP, serotonin, glutamate (e.g., NMDA receptor, AMPA receptor), GABA, acetylcholine, adenosine (e.g., A2A receptor), cannabinoid receptor, natriuretic peptide (e.g., ANP, BNP, CNP) or endorphin intracellular signaling pathway. For example, inhibition of PDE1B should act to potentiate the effect of a dopamine D1 agonist by protecting cGMP and cAMP from degradation, and should similarly inhibit dopamine D2 receptor signaling pathways, by inhibiting PDE1 activity.

Dopamine modulating agents, such as methylphenidate or medafinil, have been observed to improve cognitive function via enhanced extinction of contextual fear in animal models, and more recently, via enhancement of fear extinction learning in humans. A recent study also observed an attenuating effect of methylphenidate on the bilateral anterior insula during a fear extinction task in healthy humans. Methylphenidate and modafinil have also been shown to improve inhibitory performance and increase activation in the frontal gyrus during a stop signal task.

However, while such medications address deficiencies in cognition, they are also associated with limiting side effects. For example, commonly used drugs such as methylphenidate may be addictive to some patients. There remains a large unmet need for the treatment of cognition, including the treatment of impaired inhibitory control.

### SUMMARY OF THE DISCLOSURE

Provided herein are methods of treatment for treating a condition, disease or disorder associated with the dopamine D1 receptor intracellular pathway, e.g., a condition, disease or disorder characterized by a deficit in attention, cognition, memory and/or inhibitory processing. For example, disorders such as ADHD and PTSD are in part characterized by an impaired inhibitory processes and reactivity of the frontal gyrus, a brain region important for inhibition of response or the ability to refrain from performing a response after given a signal to stop. The inventors have unexpectedly discovered that PDE1 inhibitors as disclosed herein modulate brain activation with regional selectivity and task specificity. Administrations of the PDE1 inhibitors as disclosed herein resulted in increased activation in the frontal gyrus, similar to responses seen with methylphenidate, and consistent with improving cognition, suggesting clinical efficacy in a wide range of disorders characterized by a deficit in deficit in attention, cognition, memory and/or inhibitory processing.

Thus, in a first aspect, the present disclosure provides for a method for the prophylaxis and/or treatment of a condition, disease or disorder associated with the dopamine D1 receptor intracellular pathway, e.g., a condition, disease or disorder characterized by a deficit in attention, cognition, memory and/or inhibitory processing, the method comprising administering a pharmaceutically effective amount of a PDE1 inhibitor (i.e., a compound according to Formulas I, Ia, II, III, IV, V, and/or VI) to a subject in need thereof. In some aspects of the embodiments, the method further comprises administration of a dopamine reuptake inhibitor. In certain aspects of the embodiments, the condition, disease or disorder associated with the dopamine D1 receptor intracellular pathway is an eating disorder (e.g., anorexia nervosa, bulimia nervosa, binge eating disorder, pica, rumination disorder, avoidant/restrictive food intake disorder, purging disorder, night eating disorder, other specified feeding or eating disorder (OSFED)), a substance use disorder (e.g., addiction (e.g., a stimulant addition, such as amphetamine, cocaine, opioid, and/or nicotine), alcoholism), an obsessive-compulsive disorder (e.g., checking, contamination, mental contamination, hoarding, ruminations, intrusive thoughts, symmetry/orderliness), attention deficit hyperactivity disorder (ADHD), premature ejaculation, posttraumatic stress disorder (PTSD), a gambling disorder (e.g., gambling addiction, compulsive-pathological gambling), Tourette's syndrome and/or impulse control and conduct disorders (e.g., oppositional defiant disorder, conduct disorder, intermittent explosive disorder, kleptomania, pyromania).

In a second aspect, the present disclosure provides for a method for treating impaired inhibitory processing, the method comprising administering a pharmaceutically effective amount of a PDE1 inhibitor (i.e., a compound according to Formulas I, Ia, II, III, IV, V, and/or VI) to a patient in need thereof.

In some embodiments, the present disclosure provides for a combination therapy comprising a PDE1 inhibitor (e.g., a compound according to Formulas I, Ia, II, III, IV, V, and/or VI) and a dopamine reuptake inhibitor (e.g., methylphenidate).

### BRIEF DESCRIPTION OF THE DRAWINGS

FIG. 1 illustrates measured brain activity in human doral anterior insula during a fear extermination task following administration of a PDE1 inhibitor according to the present disclosure.

FIG. 2 illustrates measured brain activity in human inferior frontal gyms during a stop signal task following administration of a PDE1 inhibitor according to the present disclosure.

FIG. 3 illustrates measured brain activity in human dorsolateral prefrontal cortex during a stop signal task following administration of a PDE1 inhibitor according to the present disclosure.

FIG. 4 illustrates measured brain activity in human dorsal anterior cingulate cortex during a stop signal task following administration of a PDE1 inhibitor according to the present disclosure.

FIG. 5 illustrates measured brain activity in human anterior insula during a stop signal task following administration of a PDE1 inhibitor according to the present disclosure.

### DETAILED DESCRIPTION OF THE DISCLOSURE

### Compounds for Use in the Methods of the Disclosure

In one embodiment, the PDE1 inhibitors for use in the methods of treatment and prophylaxis described herein are selective PDE1 inhibitors.

### PDE1 Inhibitors

In one embodiment the invention provides that the PDE1 inhibitors for use in the methods of treatment and prophylaxis described herein are compounds of Formula I:

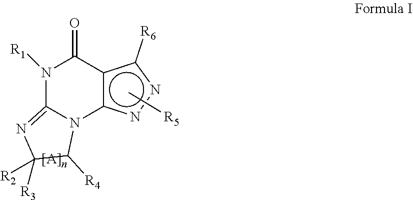

wherein
(i) R₁ is H or C₁₋₄ alkyl (e.g., methyl);
(ii) R₄ is H or C₁₋₄ alkyl and R₂ and R₃ are, independently, H or C₁₋₄ alkyl (e.g., R₂ and R₃ are both methyl, or R₂ is H and R₃ is isopropyl), aryl, heteroaryl, (optionally hetero)arylalkoxy, or (optionally hetero)arylalkyl; or R₂ is H and R₃ and R₄ together form a di-, tri- or tetramethylene bridge (pref. wherein the R₃ and R₄ together have the cis configuration, e.g., where the carbons carrying R₃ and R₄ have the R and S configurations, respectively);
(iii) R₅ is a substituted heteroarylalkyl, e.g., substituted with haloalkyl; or R₅ is attached to one of the nitrogens on the pyrazolo portion of Formula I and is a moiety of Formula A

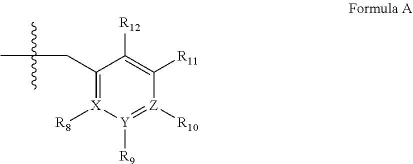

wherein X, Y and Z are, independently, N or C, and R₈, R₉, R₁₁ and R₁₂ are independently H or halogen (e.g., Cl or F), and R₁₀ is halogen, alkyl, cycloalkyl, haloalkyl (e.g., trifluoromethyl), aryl (e.g., phenyl), heteroaryl (e.g., pyridyl (for example pyrid-2-yl) optionally substituted with halogen, or thiadiazolyl (e.g., 1,2,3-thiadiazol-4-yl)), diazolyl, triazolyl, tetrazolyl, arylcarbonyl (e.g., benzoyl), alkylsulfonyl (e.g., methylsulfonyl), heteroarylcarbonyl, or alkoxycarbonyl; provided that when X, Y, or Z is nitrogen, R₈, R₉, or R₁₀, respectively, is not present; and
(iv) R₆ is H, alkyl, aryl, heteroaryl, arylalkyl (e.g., benzyl), arylamino (e.g., phenylamino), heteroarylamino, N,N-dialkylamino, N,N-diarylamino, or N-aryl-N-(arylalkyl)amino (e.g., N-phenyl-N-(1,1′-biphen-4-ylmethyl)amino); and
(v) n=0 or 1;
(vi) when n=1, A is —C(R₁₃R₁₄)—
wherein R₁₃ and R₁₄, are, independently, H or C₁₋₄ alkyl, aryl, heteroaryl, (optionally hetero)arylalkoxy or (optionally hetero)arylalkyl;
   in free, salt or prodrug form, including its enantiomers, diastereoisomers and racemates.

In another embodiment the invention provides that the PDE1 inhibitors for use in the methods as described herein are Formula 1a:

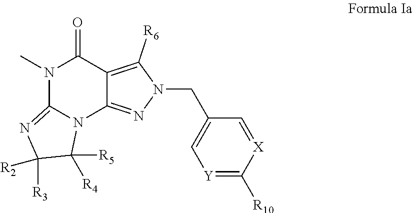

wherein
(i) R₂ and R₅ are independently H or hydroxy and R₃ and R₄ together form a tri- or tetra-methylene bridge [pref. with the carbons carrying R₃ and R₄ having the R and S configuration respectively]; or R₂ and R₃ are each methyl and R₄ and R₅ are each H; or R₂, R₄ and R₅ are H and R₃ is isopropyl [pref. the carbon carrying R₃ having the R configuration];
(ii) R₆ is (optionally halo-substituted) phenylamino, (optionally halo-substituted) benzylamino, C₁₋₄alkyl, or C₁₋₄alkyl sulfide; for example, phenylamino or 4-fluorophenylamino;
(iii) R₁₀ is C₁₋₄alkyl, methylcarbonyl, hydroxyethyl, carboxylic acid, sulfonamide, (optionally halo- or hydroxy-substituted) phenyl, (optionally halo- or hydroxy-substituted) pyridyl (for example 6-fluoropyrid-2-yl), or thiadiazolyl (e.g., 1,2,3-thiadiazol-4-yl); and X and Y are independently C or N,
in free, pharmaceutically acceptable salt or prodrug form, including its enantiomers, diastereoisomers and racemates.

In another embodiment the invention provides that the PDE1 inhibitors for use in the methods of treatment and prophylaxis described herein are compounds of Formula II:

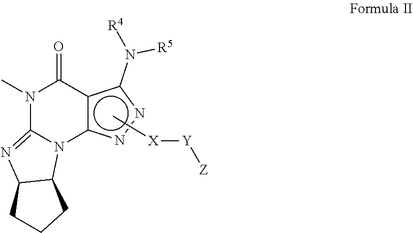

(i) X is C₁₋₆ alkylene (e.g., methylene, ethylene or prop-2-yn-1-ylene);

(ii) Y is a single bond, alkynylene (e.g., —C≡C—), arylene (e.g., phenylene) or heteroarylene (e.g., pyridylene);

(iii) Z is H, aryl (e.g., phenyl), heteroaryl (e.g., pyridyl, e.g., pyrid-2-yl), halo (e.g., F, Br, Cl), haloC₁₋₆ alkyl (e.g., trifluoromethyl), —C(O)—R¹, —N(R²)(R³), or C₃₋₇ cycloalkyl optionally containing at least one atom selected from a group consisting of N or 0 (e.g., cyclopentyl, cyclohexyl, tetrahydro-2H-pyran-4-yl, or morpholinyl);

(iv) R¹ is C₁₋₆ alkyl, haloC₁₋₆ alkyl, —OH or —OC₁₋₆ alkyl (e.g., —OCH₃);

(v) R² and R³ are independently H or C₁₋₆ alkyl;

(vi) R⁴ and R⁵ are independently H, C₁₋₆ alky or aryl (e.g., phenyl) optionally substituted with one or more halo (e.g., fluorophenyl, e.g., 4-fluorophenyl), hydroxy (e.g., hydroxyphenyl, e.g., 4-hydroxyphenyl or 2-hydroxyphenyl) or C₁₋₆ alkoxy;

(vii) wherein X, Y and Z are independently and optionally substituted with one or more halo (e.g., F, Cl or Br), C₁₋₆ alkyl (e.g., methyl), haloC₁₋₆ alkyl (e.g., trifluoromethyl), for example, Z is heteroaryl, e.g., pyridyl substituted with one or more halo (e.g., 6-fluoropyrid-2-yl, 5-fluoropyrid-2-yl, 6-fluoropyrid-2-yl, 3-fluoropyrid-2-yl, 4-fluoropyrid-2-yl, 4,6-dichloropyrid-2-yl), haloC₁₋₆ alkyl (e.g., 5-trifluoromethylpyrid-2-yl) or C₁₋₆-alkyl (e.g., 5-methylpyrid-2-yl), or Z is aryl, e.g., phenyl, substituted with one or more halo (e.g., 4-fluorophenyl), in free, salt or prodrug form.

In yet another embodiment the invention provides that the PDE1 inhibitors for use in the methods of treatment and prophylaxis described herein are Formula III:

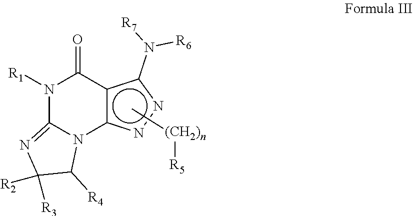

wherein
(i) R1 is H or C₁₋₄ alkyl (e.g., methyl or ethyl);
(ii) R₂ and R₃ are independently H or C₁₋₆ alkyl (e.g., methyl or ethyl); (iii) R₄ is H or C₁₋₄ alkyl (e.g., methyl or ethyl);
(iv) R₅ is aryl (e.g., phenyl) optionally substituted with one or more groups independently selected from —C(═O)—C₁₋₆ alkyl (e.g., —C(═O)—CH₃) and C₁₋₆-hydroxyalkyl (e.g., 1-hydroxyethyl);
(v) R₆ and R₇ are independently H or aryl (e.g., phenyl) optionally substituted with one or more groups independently selected from C₁₋₆ alkyl (e.g., methyl or ethyl) and halogen (e.g., F or Cl), for example unsubstituted phenyl or phenyl substituted with one or more halogen (e.g., F) or phenyl substituted with one or more C₁₋₆ alkyl and one or more halogen or phenyl substituted with one C₁₋₆ alkyl and one halogen, for example 4-fluorophenyl or 3,4-difluorophenyl or 4-fluoro-3-methylphenyl; and
(vi) n is 1, 2, 3, or 4, in free or salt form.

In yet another embodiment the invention provides that the PDE1 inhibitors for use in the methods of treatment and prophylaxis described herein are Formula IV

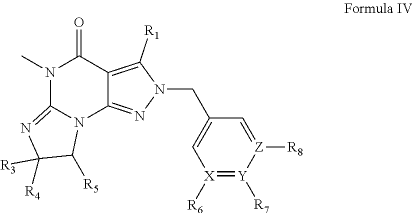

in free or salt form, wherein
(i) R₁ is C₁₋₄ alkyl (e.g., methyl or ethyl), or —NH(R₂), wherein R₂ is phenyl optionally substituted with halo (e.g., fluoro), for example, 4-fluorophenyl;
(ii) X, Y and Z are, independently, N or C;
(iii) R₃, R₄ and R₅ are independently H or C₁₋₄ alkyl (e.g., methyl); or R₃ is H and R₄ and R₅ together form a tri-methylene bridge (pref. wherein the R₄ and R₅ together have the cis configuration, e.g., where the carbons carrying R₄ and R₅ have the R and S configurations, respectively),
(iv) R₆, R₇ and R₈ are independently:
   H,
   C₁₋₄ alkyl (e.g., methyl),
   pyrid-2-yl substituted with hydroxy, or
   —S(O)₂—NH₂;
(v) Provided that when X, Y and/or Z are N, then R₆, R₇ and/or R₈, respectively, are not present; and when X, Y and Z are all C, then at least one of R₆, R₇ or R₈ is —S(O)₂—NH₂ or pyrid-2-yl substituted with hydroxy.

In another embodiment the invention provides that the PDE1 inhibitors for use in the methods as described herein are Formula V:

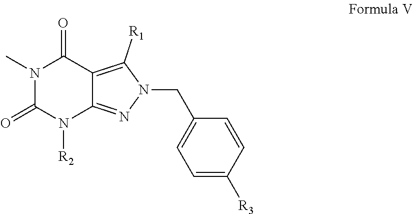

wherein
(i) R₁ is —NH(R₄), wherein R₄ is phenyl optionally substituted with halo (e.g., fluoro), for example, 4-fluorophenyl;
(ii) R₂ is H or C₁₋₆ alkyl (e.g., methyl, isobutyl or neopentyl);
(iii) R₃ is —SO₂NH₂ or —COOH;
in free or salt form.

In another embodiment the invention provides that the PDE1 inhibitors for use in the methods as described herein are Formula VI:

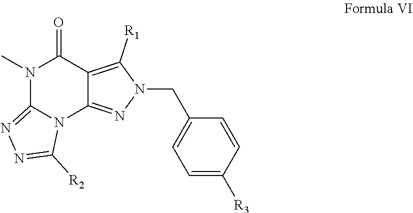

wherein
(i) R₁ is —NH(R₄), wherein R₄ is phenyl optionally substituted with halo (e.g., fluoro), for example, 4-fluorophenyl;
(ii) R₂ is H or C₁₋₆ alkyl (e.g., methyl or ethyl);
(iii) R₃ is H, halogen (e.g., bromo), C₁₋₆ alkyl (e.g., methyl), aryl optionally substituted with halogen (e.g., 4-fluorophenyl), heteroaryl optionally substituted with halogen (e.g., 6-fluoropyrid-2-yl or pyrid-2-yl), or acyl (e.g., acetyl),
in free or salt form.

In one embodiment, the present disclosure provides for administration of a PDE1 inhibitor for use in the methods described herein (e.g., a compound according to Formulas I, Ia, II, III, IV, V, and/or VI), wherein the inhibitor is a compound according to the following:

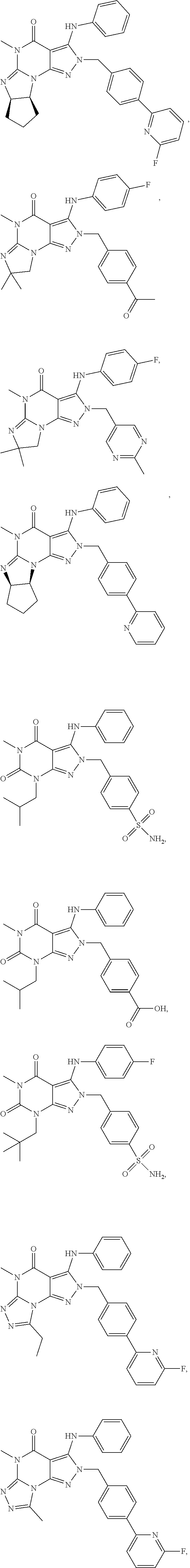

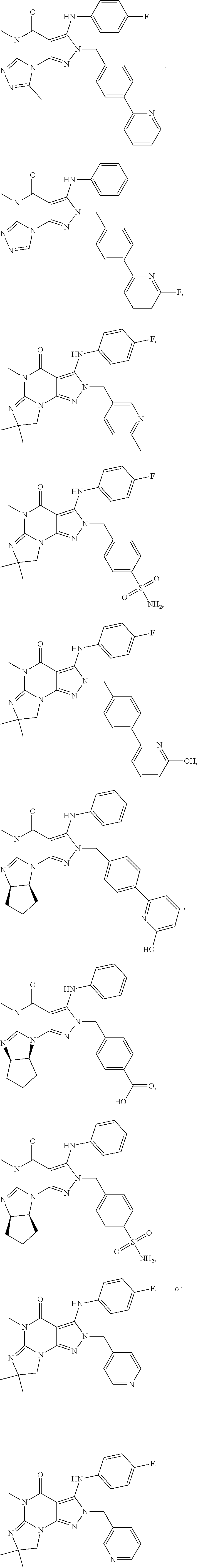

In one embodiment the invention provides administration of a PDE1 inhibitor for treatment or prophylaxis of inflammation or an inflammatory related disease or disorder, wherein the inhibitor is a compound according to the following:

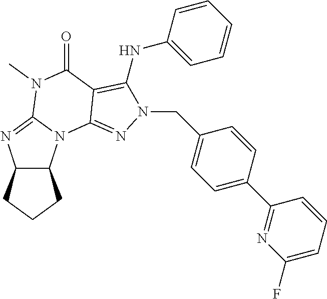 [SMILES: CN1C(=O)c2c(nn(Cc3ccc(-c4cccc(F)n4)cc3)c2Nc2ccccc2)N2C1=N[C@@H]1CCC[C@@H]12]

in free or pharmaceutically acceptable salt form.

In still another embodiment, the invention provides administration of a PDE1 inhibitor for treatment or prophylaxis of inflammation or an inflammatory related disease or disorder, wherein the inhibitor is a compound according to the following:

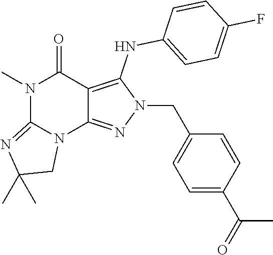 [SMILES: CC(=O)c1ccc(Cn2nc3c(c2Nc2ccc(F)cc2)C(=O)N(C)C2=NC(C)(C)CN23)cc1]

in free or pharmaceutically acceptable salt form.

In still another embodiment, the invention provides administration of a PDE1 inhibitor for treatment or prophylaxis of inflammation or an inflammatory related disease or disorder, wherein the inhibitor is a compound according to the following:

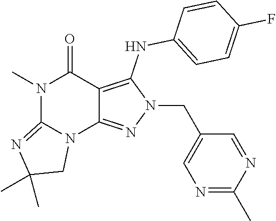 [SMILES: Cc1ncc(Cn2nc3c(c2Nc2ccc(F)cc2)C(=O)N(C)C2=NC(C)(C)CN23)cn1]

in free or pharmaceutically acceptable salt form.

In still another embodiment, the invention provides administration of a PDE1 inhibitor for treatment or prophylaxis of inflammation or an inflammatory related disease or disorder, wherein the inhibitor is a compound according to the following:

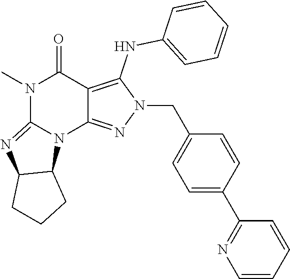 [SMILES: CN1C(=O)c2c(nn(Cc3ccc(-c4ccccn4)cc3)c2Nc2ccccc2)N2C1=N[C@@H]1CCC[C@@H]12]

in free or pharmaceutically acceptable salt form.

In still another embodiment, the invention provides administration of a PDE1 inhibitor for treatment or prophylaxis of inflammation or an inflammatory related disease or disorder, wherein the inhibitor is a compound according to the following:

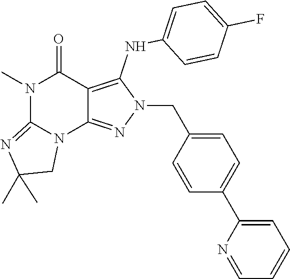 [SMILES: CN1C(=O)c2c(nn(Cc3ccc(-c4ccccn4)cc3)c2Nc2ccc(F)cc2)N2CC(C)(C)N=C12]

in free or pharmaceutically acceptable salt form.

In one embodiment, selective PDE1 inhibitors of any of the preceding formulae (e.g., Formulas I, Ia, II, III, IV, V, and/or VI) are compounds that inhibit phosphodiesterase-mediated (e.g., PDE1-mediated, especially PDE1B-mediated) hydrolysis of cGMP, e.g., the preferred compounds have an IC50 of less than 1μM, preferably less than 500 nM, preferably less than 50 nM, and preferably less than 5 nM in an immobilized-metal affinity particle reagent PDE assay, in free or salt form.

In other embodiments, the invention provides administration of a PDE1 inhibitor for treatment according to the methods described herein, wherein the inhibitor is a compound according to the following:

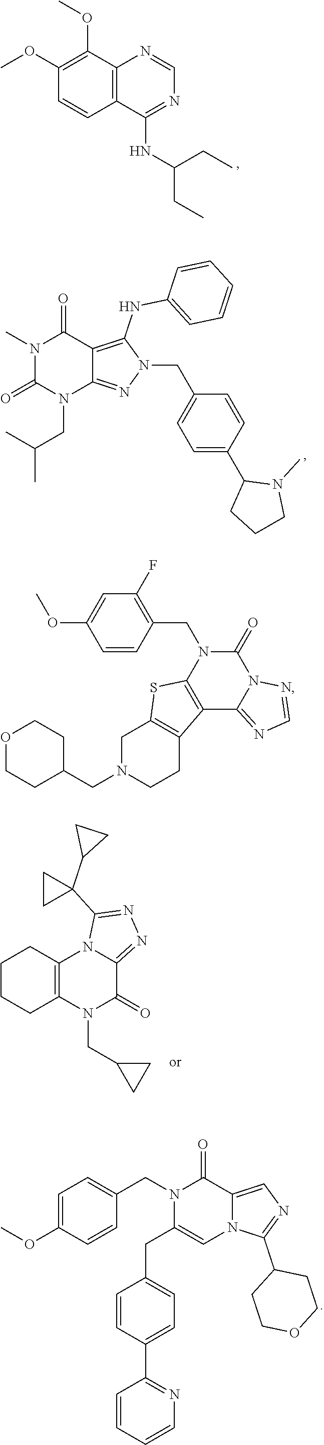

Further examples of PDE1 inhibitors suitable for use in the methods and treatments discussed herein can be found in International Publication WO2006133261A2; U.S. Pat. Nos. 8,273,750; 9,000,001; 9,624,230; International Publication WO2009075784A1; U.S. Pat. Nos. 8,273,751; 8,829,008; 9,403,836; International Publication WO2014151409A1, U.S. Pat. Nos. 9,073,936; 9,598,426; U.S. Patent 9,556,186; U.S. Publication 2017/0231994A1, International Publication WO2016022893A1, and U.S. Publication 2017/0226117A1, each of which are incorporated by reference in their entirety.

Still further examples of PDE1 inhibitors suitable for use in the methods and treatments discussed herein can be found in International Publication WO2018007249A1; U.S. Publication 2018/0000786; International Publication WO2015118097A1; U.S. Pat. No. 9,718,832; International Publication WO2015091805A1; U.S. Pat. No. 9,701,665; U.S. Publication 2015/0175584A1; U.S. Publication 2017/0267664A1; International Publication WO2016055618A1; U.S. Publication 2017/0298072A1; International Publication WO2016170064A1; U.S. Publication 2016/0311831A1; International Publication WO2015150254A1; U.S. Publication 2017/0022186A1; International Publication WO2016174188A1; U.S. Publication 2016/0318939A1; U.S. Publication 2017/0291903A1; International Publication WO2018073251A1; International Publication WO2017178350A1; U.S. Publication 2017/0291901A1; International Publication WO2018/115067; U.S. Publication 2018/0179200A; U.S. Publication US20160318910A1; U.S. Pat. No. 9,868,741; International Publication WO2017/139186A1; International Application WO2016/040083; U.S. Publication 2017/0240532; International Publication WO 2016033776A1; U.S. Publication 2017/0233373; International Publication WO2015130568; International Publication WO2014159012; U.S. Pat. Nos. 9,034,864; 9,266,859; International Publication WO2009085917; U.S. Pat. No. 8,084,261; International Publication WO2018039052; U.S. Publication US20180062729; and International Publication WO2019027783 each of which are incorporated by reference in their entirety. In any situation in which the statements of any documents incorporated by reference contradict or are incompatible with any statements made in the present disclosure, the statements of the present disclosure shall be understood as controlling.

Still further examples of PDE1 inhibitors and suitable methods of use are disclosed in International Application PCT/US2019/033941 and U.S. Provisional Application 62/789,499, both of which are incorporated by reference herein.

If not otherwise specified or clear from context, the following terms herein have the following meanings:
(a) “Selective PDE1 inhibitor” as used herein refers to a PDE1 inhibitor with at least 100-fold selectivity for PDE1 inhibition over inhibition of any other PDE isoform.
(b) “Alkyl” as used herein is a saturated or unsaturated hydrocarbon moiety, preferably saturated, preferably having one to six carbon atoms, which may be linear or branched, and may be optionally mono-, di- or tri-substituted, e.g., with halogen (e.g., chloro or fluoro), hydroxy, or carboxy.
(c) “Cycloalkyl” as used herein is a saturated or unsaturated nonaromatic hydrocarbon moiety, preferably saturated, preferably comprising three to nine carbon atoms, at least some of which form a nonaromatic mono- or bicyclic, or bridged cyclic structure, and which may be optionally substituted, e.g., with halogen (e.g., chloro or fluoro), hydroxy, or carboxy. Wherein the cycloalkyl optionally contains one or more atoms selected from N and 0 and/or S, said cycloalkyl may also be a heterocycloalkyl.
(d) “Heterocycloalkyl” is, unless otherwise indicated, saturated or unsaturated nonaromatic hydrocarbon moiety, preferably saturated, preferably comprising three to nine carbon atoms, at least some of which form a nonaromatic mono- or bicyclic, or bridged cyclic structure, wherein at least one carbon atom is replaced with N, O or S, which heterocycloalkyl may be optionally substituted, e.g., with halogen (e.g., chloro or fluoro), hydroxy, or carboxy.
(e) “Aryl” as used herein is a mono or bicyclic aromatic hydrocarbon, preferably phenyl, optionally substituted, e.g., with alkyl (e.g., methyl), halogen (e.g., chloro or fluoro), haloalkyl (e.g., trifluoromethyl), hydroxy, carboxy, or an additional aryl or heteroaryl (e.g., biphenyl or pyridylphenyl).
(f) “Heteroaryl” as used herein is an aromatic moiety wherein one or more of the atoms making up the aromatic ring is sulfur or nitrogen rather than carbon, e.g., pyridyl or thiadiazolyl, which may be optionally substituted, e.g., with alkyl, halogen, haloalkyl, hydroxy or carboxy.

Compounds of the Disclosure, e.g., PDE1 inhibitors as described herein, may exist in free or salt form, e.g., as acid addition salts. In this specification unless otherwise indicated, language such as “Compounds of the Disclosure” is to be understood as embracing the compounds in any form, for example free or acid addition salt form, or where the compounds contain acidic substituents, in base addition salt form. The Compounds of the Disclosure are intended for use as pharmaceuticals, therefore pharmaceutically acceptable salts are preferred. Salts which are unsuitable for pharmaceutical uses may be useful, for example, for the isolation or purification of free Compounds of the Disclosure or their pharmaceutically acceptable salts, are therefore also included.

Compounds of the Disclosure may in some cases also exist in prodrug form. A prodrug form is compound which converts in the body to a Compound of the Disclosure. For example, when the Compounds of the Disclosure contain hydroxy or carboxy substituents, these substituents may form physiologically hydrolysable and acceptable esters. As used herein, “physiologically hydrolysable and acceptable ester” means esters of Compounds of the Disclosure which are hydrolysable under physiological conditions to yield acids (in the case of Compounds of the Disclosure which have hydroxy substituents) or alcohols (in the case of Compounds of the Disclosure which have carboxy substituents) which are themselves physiologically tolerable at doses to be administered. Therefore, wherein the Compound of the Disclosure contains a hydroxy group, for example, Compound-OH, the acyl ester prodrug of such compound, i.e., Compound-O—C(O)—C₁₋₄ alkyl, can hydrolyze in the body to form physiologically hydrolysable alcohol (Compound-OH) on the one hand and acid on the other (e.g., HOC(O)—C₁₋₄ alkyl). Alternatively, wherein the Compound of the Disclosure contains a carboxylic acid, for example, Compound-C(O)OH, the acid ester prodrug of such compound, Compound-C(O)O—C₁₋₄alkyl can hydrolyze to form Compound-C(O)OH and HO—C₁₋₄alkyl. As will be appreciated the term thus embraces conventional pharmaceutical prodrug forms.

In another embodiment, the disclosure further provides a pharmaceutical composition comprising a PDE1 inhibitor in combination with a dopamine reuptake inhibitor, each in free or pharmaceutically acceptable salt form, in admixture with a pharmaceutically acceptable carrier. The term “combination,” as used herein, embraces simultaneous, sequential, or contemporaneous administration of the PDE1 inhibitor and the dopamine reuptake inhibitor. In another embodiment, the disclosure provides a pharmaceutical composition containing such a compound. In some embodiments, the combination of the PDE1 inhibitor and the dopamine reuptake inhibitor allows the dopamine reuptake inhibitor to be administered in a dosage lower than would be effective if administered as sole monotherapy.

### Methods of Using Compounds of the Disclosure

In another embodiment, the present application provides for a method [Method 1] for the prophylaxis and/or treatment of a condition, disease or disorder associated with the dopamine D1 receptor intracellular pathway, e.g., a condition, disease or disorder characterized by a deficit in attention, cognition, memory and/or inhibitory processing, the method comprising administering a pharmaceutically effective amount of a PDE1 inhibitor (i.e., a compound according to Formulas I, Ia, II, III, IV, V, and/or VI) to a subject in need thereof.

Further methods are provided as follows:
1.1 Method 1, wherein the condition, disease or disorder is characterized by a deficit in attention, cognition, memory and/or inhibitory processing.
1.2 Any preceding method, wherein the condition, disease or disorder is selected from an eating disorder (e.g., anorexia nervosa, bulimia nervosa, binge eating disorder, pica, rumination disorder, avoidant/restrictive food intake disorder, purging disorder, night eating disorder, other specified feeding or eating disorder (OSFED)), a substance use disorder (e.g., addiction (e.g., a stimulant addition, such as amphetamine, cocaine, opiate, and/or nicotine), alcoholism), an obsessive-compulsive disorder (e.g., checking, contamination, mental contamination, hoarding, ruminations, intrusive thoughts, symmetry/orderliness), attention deficit hyperactivity disorder (ADHD), premature ejaculation, posttraumatic stress disorder (PTSD), a gambling disorder (e.g., gambling addiction, compulsive-pathological gambling), Tourette's syndrome and/or impulse control and conduct disorders (e.g., oppositional defiant disorder, conduct disorder, intermittent explosive disorder, kleptomania, pyromania).
1.3 Any preceding method, wherein the condition, disease or disorder is an eating disorder (e.g., anorexia nervosa, bulimia nervosa, binge eating disorder, pica, rumination disorder, avoidant/restrictive food intake disorder, purging disorder, night eating disorder, other specified feeding or eating disorder (OSFED)).
1.4 The preceding method, wherein the eating disorder is anorexia nervosa, bulimia nervosa, binge eating disorder, pica, rumination disorder, avoidant/restrictive food intake disorder, purging disorder, night eating disorder, other specified feeding and/or eating disorder (OSFED).
1.5 Any preceding method, wherein the condition, disease or disorder is a substance use disorder (e.g., addiction (e.g., a stimulant addition, such as amphetamine, cocaine, opiate, and/or nicotine), alcoholism).
1.6 The preceding method, wherein the substance abuse disorder is addiction (e.g., a stimulant addition, such as amphetamine, cocaine, opiate, and/or nicotine) or alcoholism.
1.7 Any preceding method, wherein the condition, disease or disorder is an obsessive-compulsive disorder (OCD) (e.g., checking OCD, contamination OCD, mental contamination OCD, hoarding OCD, ruminations OCD, intrusive thoughts OCD, symmetry/orderliness OCD).
1.8 The preceding method, wherein the obsessive-compulsive disorder is checking OCD, contamination OCD, mental contamination OCD, hoarding OCD, ruminations OCD, intrusive thoughts OCD, and/or symmetry/orderliness OCD.
1.9 Any preceding method, wherein the condition, disease or disorder is attention deficit hyperactivity disorder (ADHD).
1.10 Any preceding method, wherein the condition, disease or disorder is premature ejaculation.
1.11 Any preceding method, wherein the condition, disease or disorder is posttraumatic stress disorder (PTSD).
1.12 Any preceding method, wherein the condition, disease or disorder is a gambling disorder (e.g., gambling addiction, compulsive-pathological gambling).
1.13 The preceding method, wherein the gambling disorder is gambling addiction or compulsive-pathological gambling.
1.14 Any preceding method, wherein the condition, disease or disorder is Tourette's syndrome.
1.15 Any preceding method, wherein the condition, disease or disorder is impulse control and conduct disorders (e.g., oppositional defiant disorder, conduct disorder, intermittent explosive disorder, kleptomania, pyromania).
1.16 The preceding method, wherein the impulse control and conduct disorder is oppositional defiant disorder, conduct disorder, intermittent explosive disorder, kleptomania and/or pyromania.
1.17 Any of the preceding methods, wherein the PDE1 inhibitor is administered in an amount of about 0.01 to about 20 mg/kg, e.g. about 0.1 to about 5 mg/kg, e.g. about 1 mg/kg.
1.18 Any of the preceding methods, wherein the PDE1 inhibitor is administered orally.
1.19 Any of the preceding methods, wherein the PDE1 inhibitor is administered via injection.
1.20 Any of the preceding methods, wherein the PDE1 inhibitor is administered with an additional therapeutic agent.
1.21 Any of the preceding methods, wherein the PDE1 inhibitor is administered with a monoamine reuptake inhibitor (e.g., a dopamine reuptake inhibitor, a serotonin reuptake inhibitor and/or a norepinephrine reuptake inhibitor).
1.22 Any of the preceding methods, wherein the PDE1 inhibitor is administered with one or more of 4-Hydroxy-1-methyl-4-(4-methylphenyl)-3-piperidyl-4-methylphenyl ketone, adrafinil, amantadine, atomoxetine, amfonelic acid, amineptine, amitriptyline, amoxapine, benztropine, bupropion, hydroxybupropion, butriptyline, chlorphenamine, citalopram, clomipramine, cocaethylene, desipramine, desmethylcitalopram, desmethylsertraline, desmethylsibutramine, desoxypipradrol, desvenlafaxine, dextroamphetamine, dextromethamphetamine, didesmethylsibutramine, diphenhydramine, dosulepin, doxepin, duloxetine, escitalopram, etoperidone, femoxetine, fluorenol, fluoxetine, fluvoxamine, GBR-12783, GBR-12935, GBR-13069, GBR-13098, GYKI-52895, imipramine, indatraline, iprindole, iometopane, levomilnacipran, lofepramine, maprotiline, mazindol, medifoxamine, metaphit, methylenedioxypyrovalerone (MDPV), methylphenidate, ethylphenidate, mianserin, milnacipran, mirtazapine, modafinil, armodafinil, nefazodone, nefopam, nisoxetine, nomifensine, norfluoxetine, nortriptyline, oxaprotiline, paroxetine, protriptyline, reboxetine, rimcazole, RTI-229, sertraline, sibutramine, trazodone, trimipramine, vanoxerine, venlafaxine, vilazodone, viloxazine, vortioxetine, zimelidine, Chaenomeles speciose extract, and/or Oroxylin A.
1.23 Any of the preceding methods, wherein the PDE1 inhibitor is administered with one or more of atomoxetine, reboxetine, nisoxetine, desvenlafaxine, venlafaxine, duloxetine, milnacipran, fluoxetine, sertraline, citalopram, escitalopram, bupropion, and/or nomifensine.
1.24 Any of the preceding methods, wherein the PDE1 inhibitor is administered with a dopamine reuptake inhibitor.
1.25 Any of the preceding methods, wherein the PDE1 inhibitor is administered with a dopamine reuptake inhibitor selected from 4-Hydroxy-1-methyl-4-(4-methylphenyl)-3-piperidyl-4-methylphenyl ketone, altropane, amfonelic acid, amineptine, BTCP, 3C-PEP, DBL-583, difluoropine, GBR-12783, GBR-12935, GBR-13069, GBR-13098, GYKI-52895, iometopane, methylphenidate, ethylphenidate, modafinil, armodafinil, RTI-229, vanoxerine, adrafinil, amantadine, benztropine, bupropion, fluorenol, medifoxamine, metaphit, rimcazole, venlafaxine, Chaenomeles speciose extract, Oroxylin A, or combinations thereof.
1.26 Any of the preceding methods, wherein the PDE1 inhibitor is administered with a norepinephrine and serotonin reuptake inhibitor.
1.27 Any of the preceding methods, wherein the PDE1 inhibitor is administered with a norepinephrine and serotonin reuptake inhibitor according to Formula VII:

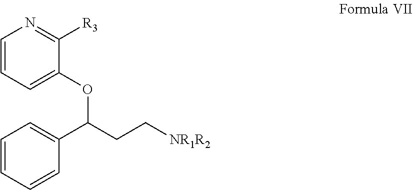

in free or salt form, wherein:
   R₁ and R₂ are independently H or C₁₋₄ alkyl (e.g., methyl or ethyl); and
   R₃ is n-C₂₋₄ alkyl (e.g., ethyl or n-propyl), or —O—C₁₋₄ alkyl (e.g., methoxy or ethoxy) optionally substituted hydroxy.
1.28 Any of the preceding methods, wherein the PDE1 inhibitor is administered with a norepinephrine and serotonin reuptake inhibitor according to

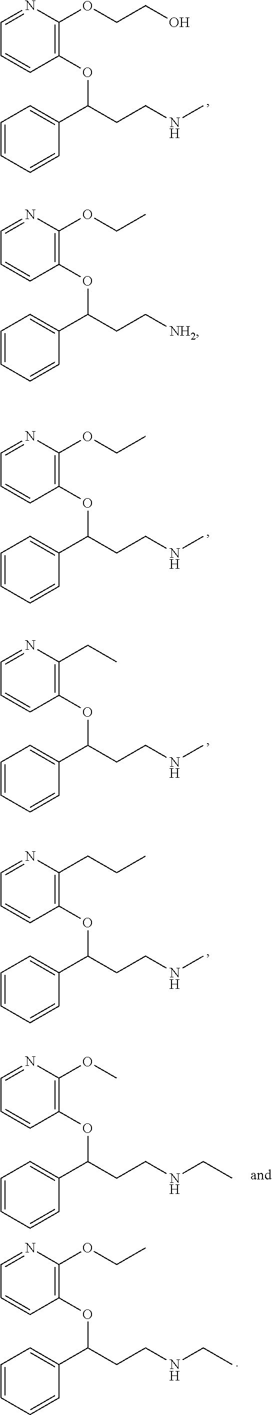

1.29 Any of the preceding methods, wherein the PDE1 inhibitor is a compound according to any of Formulas I, Ia, II, III, IV, V, and/or VI.
1.30 Any of the preceding methods, wherein the PDE1 inhibitor is a compound according to Formula Ia.
1.31 Any of the preceding methods, wherein the PDE1 inhibitor is a compound according to:

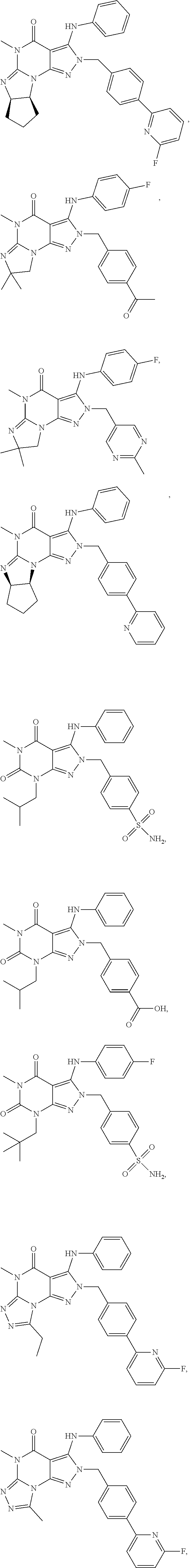

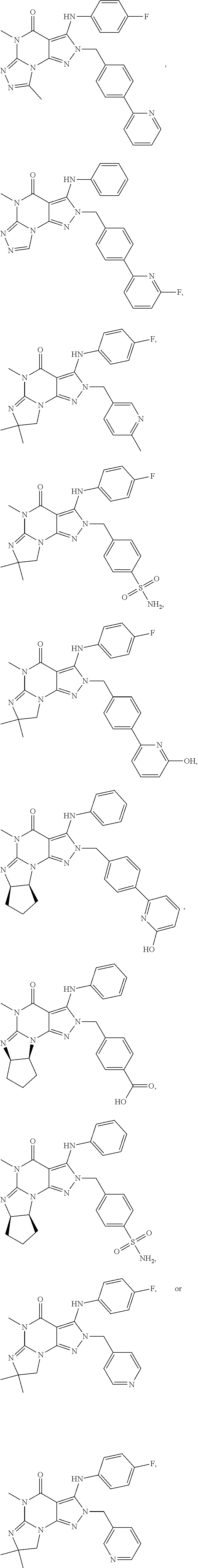

in free or pharmaceutically acceptable salt form.
1.32 Any of the preceding methods, wherein the PDE1 inhibitor is a compound according to:

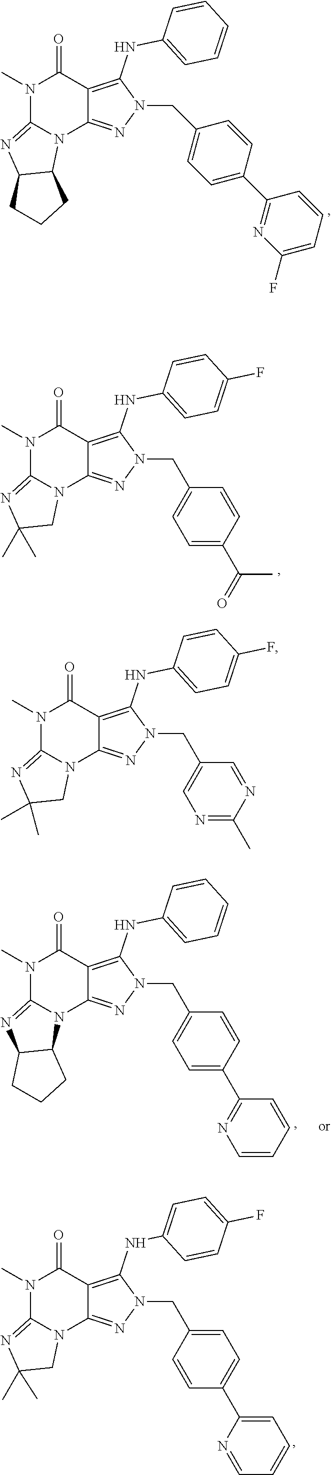

in free or pharmaceutically acceptable salt form.
1.33 Any of the preceding methods, wherein the PDE1 inhibitor is a compound according to:

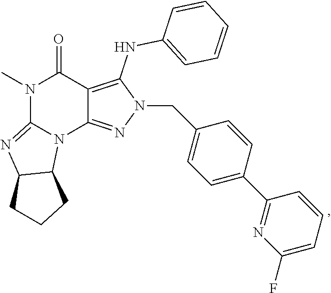 [SMILES: CN1C(=O)c2c(nn(Cc3ccc(-c4cccc(F)n4)cc3)c2Nc2ccccc2)N2C1=N[C@@H]1CCC[C@@H]12]

in free or pharmaceutically acceptable salt form.
1.34 Any of the preceding methods, wherein the PDE1 inhibitor is a compound according to:

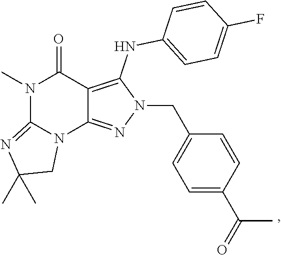 [SMILES: CC(=O)c1ccc(Cn2nc3c(c2Nc2ccc(F)cc2)C(=O)N(C)C2=NC(C)(C)CN23)cc1]

in free or pharmaceutically acceptable salt form.
1.35 Any of the preceding methods, wherein the PDE1 inhibitor is a compound according to:

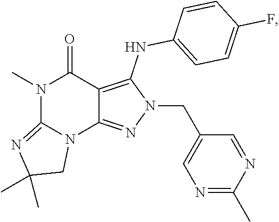 [SMILES: Cc1ncc(Cn2nc3c(c2Nc2ccc(F)cc2)C(=O)N(C)C2=NC(C)(C)CN23)cn1]

in free or pharmaceutically acceptable salt form.
1.36 Any of the preceding methods, wherein the PDE1 inhibitor is a compound according to:

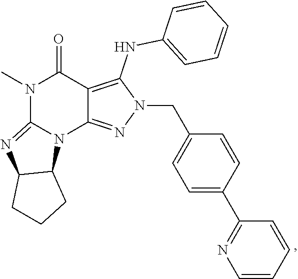 [SMILES: CN1C(=O)c2c(nn(Cc3ccc(-c4ccccn4)cc3)c2Nc2ccccc2)N2C1=N[C@@H]1CCC[C@@H]12]

in free or pharmaceutically acceptable salt form.
1.37 Any of the preceding methods, wherein the PDE1 inhibitor is a compound according to:

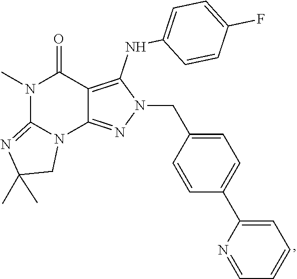 [SMILES: CN1C(=O)c2c(nn(Cc3ccc(-c4ccccn4)cc3)c2Nc2ccc(F)cc2)N2CC(C)(C)N=C12]

in free or pharmaceutically acceptable salt form.
1.38 Any of the preceding methods, wherein the patient is a human.

The disclosure further provides a PDE1 inhibitor for use in a method for the prophylaxis and/or treatment of a condition, disease or disorder associated with the dopamine D1 receptor intracellular pathway, e.g., a condition, disease or disorder characterized by a deficit in attention, cognition, memory and/or inhibitory processing, e.g., for use in any of Methods 1, et seq.

The disclosure further provides the use of a PDE1 inhibitor in the manufacture of a medicament for use in a method for the prophylaxis and/or treatment of a condition, disease or disorder associated with the dopamine D1 receptor intracellular pathway, e.g., a condition, disease or disorder characterized by a deficit in attention, cognition, memory and/or inhibitory processing, e.g., a medicament for use in any of Methods 1, et seq.

The invention further provides a pharmaceutical composition comprising a PDE1 inhibitor, e.g., any of a Compound of Formulas I, Ia, II, III, IV, V, and/or VI, for use in any of Methods 1, et seq.

In another embodiment, the present application provides for a method (Method 2) for treating impaired inhibitory processing, the method comprising administering a pharmaceutically acceptable amount of a PDE1 inhibitor (i.e., PDE1 inhibitor according to Formulas I, Ia, II, III, IV, V, and/or VI) to a patient in need thereof.

Further methods are provided as follows:
2.1 Method 2, wherein the patient is suffering from a condition, disease or disorder associated with the dopamine D1 receptor intracellular pathway.
2.2 Any preceding method, wherein the patient is suffering from condition, disease or disorder selected from an eating disorder (e.g., anorexia nervosa, bulimia nervosa, binge eating disorder, pica, rumination disorder, avoidant/restrictive food intake disorder, purging disorder, night eating disorder, other specified feeding or eating disorder (OSFED)), a substance use disorder (e.g., addiction (e.g., a stimulant addition, such as amphetamine, cocaine, opiate, and/or nicotine), alcoholism), an obsessive-compulsive disorder (e.g., checking, contamination, mental contamination, hoarding, ruminations, intrusive thoughts, symmetry/orderliness), attention deficit hyperactivity disorder (ADHD), premature ejaculation, posttraumatic stress disorder (PTSD), a gambling disorder (e.g., gambling addiction, compulsive-pathological gambling), Tourette's syndrome and/or impulse control and conduct disorders (e.g., oppositional defiant disorder, conduct disorder, intermittent explosive disorder, kleptomania, pyromania).
2.3 Any preceding method, wherein the patient is suffering from an eating disorder (e.g., anorexia nervosa, bulimia nervosa, binge eating disorder, pica, rumination disorder, avoidant/restrictive food intake disorder, purging disorder, night eating disorder, other specified feeding or eating disorder (OSFED)).
2.4 The preceding method, wherein the eating disorder is anorexia nervosa, bulimia nervosa, binge eating disorder, pica, rumination disorder, avoidant/restrictive food intake disorder, purging disorder, night eating disorder, other specified feeding and/or eating disorder (OSFED).
2.5 Any preceding method, wherein the patient is suffering from a substance use disorder (e.g., addiction (e.g., a stimulant addition, such as amphetamine, cocaine, opiate, and/or nicotine), alcoholism).
2.6 The preceding method, wherein the substance abuse disorder is addiction (e.g., a stimulant addition, such as amphetamine, cocaine, opiate, and/or nicotine) or alcoholism.
2.7 Any preceding method, wherein the patient is suffering from an obsessive-compulsive disorder (OCD) (e.g., checking OCD, contamination OCD, mental contamination OCD, hoarding OCD, ruminations OCD, intrusive thoughts OCD, symmetry/orderliness OCD).
2.8 The preceding method, wherein the obsessive-compulsive disorder is checking OCD, contamination OCD, mental contamination OCD, hoarding OCD, ruminations OCD, intrusive thoughts OCD, and/or symmetry/orderliness OCD.
2.9 Any preceding method, wherein the patient is suffering from attention deficit hyperactivity disorder (ADHD).
2.10 Any preceding method, wherein the patient is suffering from premature ejaculation.
2.11 Any preceding method, wherein the patient is suffering from posttraumatic stress disorder (PTSD).
2.12 Any preceding method, wherein the patient is suffering from a gambling disorder (e.g., gambling addiction, compulsive-pathological gambling).
2.13 The preceding method, wherein the gambling disorder is gambling addiction or compulsive-pathological gambling.
2.14 Any preceding method, wherein the condition, disease or disorder is Tourette's syndrome.
2.15 Any preceding method, wherein the patient is suffering from impulse control and conduct disorders (e.g., oppositional defiant disorder, conduct disorder, intermittent explosive disorder, kleptomania, pyromania).
2.16 The preceding method, wherein the impulse control and conduct disorder is oppositional defiant disorder, conduct disorder, intermittent explosive disorder, kleptomania and/or pyromania.
2.17 Any of the preceding methods, wherein the PDE1 inhibitor is administered in an amount of about 0.01 to about 20 mg/kg, e.g. about 0.1 to about 5 mg/kg, e.g. about 1 mg/kg.
2.18 Any of the preceding methods, wherein the PDE1 inhibitor is administered orally.
2.19 Any of the preceding methods, wherein the PDE1 inhibitor is administered via injection.
2.20 Any of the preceding methods, wherein the PDE1 inhibitor is administered with an additional therapeutic agent.
2.21 Any of the preceding methods, wherein the PDE1 inhibitor is administered with a monoamine reuptake inhibitor (e.g., a dopamine reuptake inhibitor, a serotonin reuptake inhibitor and/or a norepinephrine reuptake inhibitor).
2.22 Any of the preceding methods, wherein the PDE1 inhibitor is administered with one or more of 4-Hydroxy-1-methyl-4-(4-methylphenyl)-3-piperidyl-4-methylphenyl ketone, adrafinil, amantadine, atomoxetine, amfonelic acid, amineptine, amitriptyline, amoxapine, benztropine, bupropion, hydroxybupropion, butriptyline, chlorphenamine, citalopram, clomipramine, cocaethylene, desipramine, desmethylcitalopram, desmethylsertraline, desmethylsibutramine, desoxypipradrol, desvenlafaxine, dextroamphetamine, dextromethamphetamine, didesmethylsibutramine, diphenhydramine, dosulepin, doxepin, duloxetine, escitalopram, etoperidone, femoxetine, fluorenol, fluoxetine, fluvoxamine, GBR-12783, GBR-12935, GBR-13069, GBR-13098, GYKI-52895, imipramine, indatraline, iprindole, iometopane, levomilnacipran, lofepramine, maprotiline, mazindol, medifoxamine, metaphit, methylenedioxypyrovalerone (MDPV), methylphenidate, ethylphenidate, mianserin, milnacipran, mirtazapine, modafinil, armodafinil, nefazodone, nefopam, nisoxetine, nomifensine, norfluoxetine, nortriptyline, oxaprotiline, paroxetine, protriptyline, reboxetine, rimcazole, RTI-229, sertraline, sibutramine, trazodone, trimipramine, vanoxerine, venlafaxine, vilazodone, viloxazine, vortioxetine, zimelidine, Chaenorneles speciose extract, and/or Oroxylin A.
2.23 Any of the preceding methods, wherein the PDE1 inhibitor is administered with one or more of atomoxetine, reboxetine, nisoxetine, desvenlafaxine, venlafaxine, duloxetine, milnacipran, fluoxetine, sertraline, citalopram, escitalopram, bupropion, and/or nomifensine.
2.24 Any of the preceding methods, wherein the PDE1 inhibitor is administered with a dopamine reuptake inhibitor.
2.25 Any of the preceding methods, wherein the PDE1 inhibitor is administered with a dopamine reuptake inhibitor selected from 4-Hydroxy-1-methyl-4-(4-methylphenyl)-3-piperidyl-4-methylphenyl ketone, altropane, amfonelic acid, amineptine, BTCP, 3C-PEP, DBL-583, difluoropine, GBR-12783, GBR-12935, GBR-13069, GBR-13098, GYKI-52895, iometopane, methylphenidate, ethylphenidate, modafinil, armodafinil, RTI-229, vanoxerine, adrafinil, amantadine, benztropine, bupropion, fluorenol, medifoxamine, metaphit, rimcazole, venlafaxine, Chaenomeles speciose extract, Oroxylin A, or combinations thereof.
2.26 Any of the preceding methods, wherein the PDE1 inhibitor is administered with a norepinephrine and serotonin reuptake inhibitor.
2.27 Any of the preceding methods, wherein the PDE1 inhibitor is administered with a norepinephrine and serotonin reuptake inhibitor according to Formula VII:

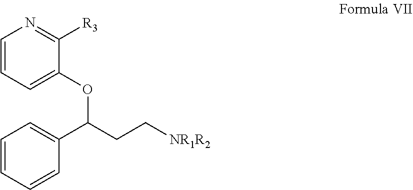

in free or salt form, wherein:
   R₁ and R₂ are independently H or C₁₋₄ alkyl (e.g., methyl or ethyl); and
   R₃ is n-C₂₋₄ alkyl (e.g., ethyl or n-propyl), or —O—C₁₋₄ alkyl (e.g., methoxy
   or ethoxy) optionally substituted hydroxy.
2.28 Any of the preceding methods, wherein the PDE1 inhibitor is administered with a norepinephrine and serotonin reuptake inhibitor according to

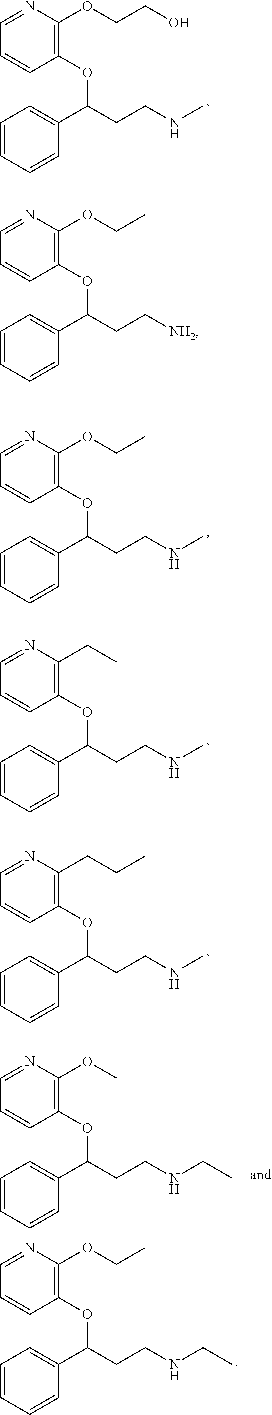

2.29 Any of the preceding methods, wherein the PDE1 inhibitor is a compound according to any of Formulas I, Ia, II, III, IV, V, and/or VI.
2.30 Any of the preceding methods, wherein the PDE1 inhibitor is a compound according to Formula Ia.
2.31 Any of the preceding methods, wherein the PDE1 inhibitor is a compound according to:

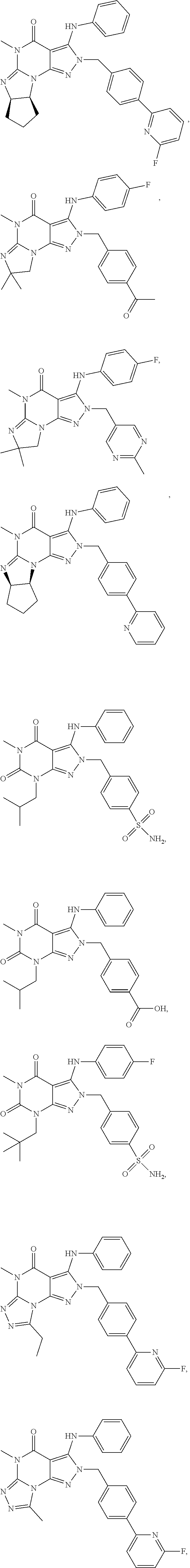

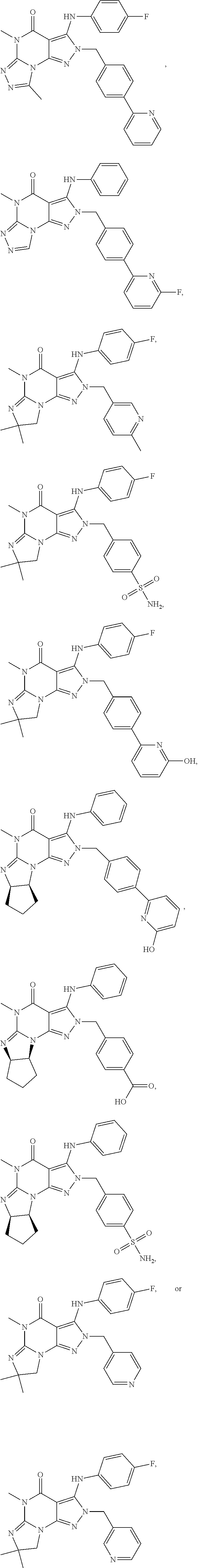

in free or pharmaceutically acceptable salt form.
2.32 Any of the preceding methods, wherein the PDE1 inhibitor is a compound according to:

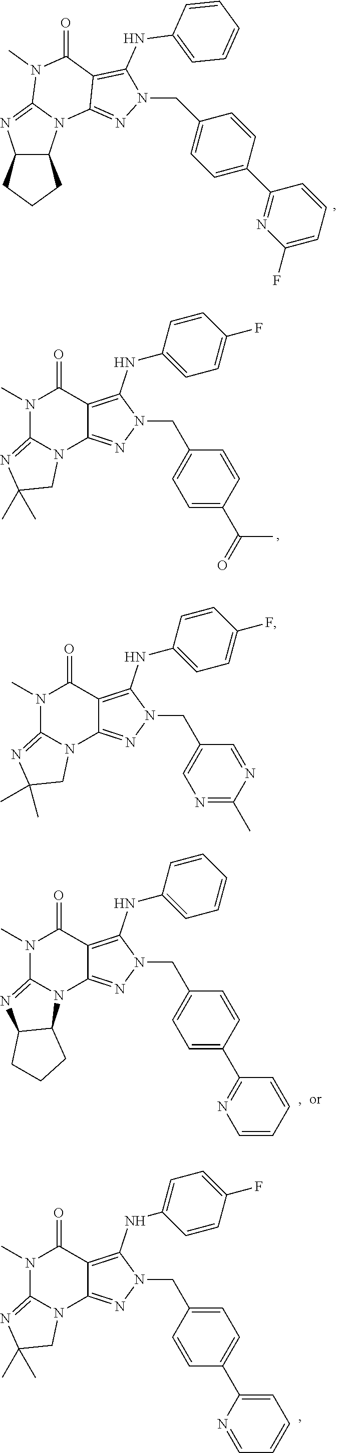

in free or pharmaceutically acceptable salt form.
2.33 Any of the preceding methods, wherein the PDE1 inhibitor is a compound according to:

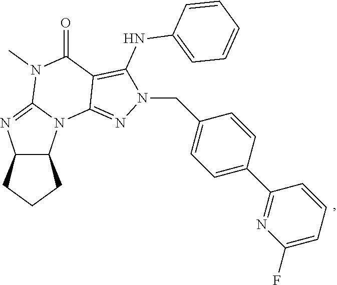 [SMILES: CN1C(=O)c2c(nn(Cc3ccc(-c4cccc(F)n4)cc3)c2Nc2ccccc2)N2C1=N[C@@H]1CCC[C@@H]12]

in free or pharmaceutically acceptable salt form.
2.34 Any of the preceding methods, wherein the PDE1 inhibitor is a compound according to:

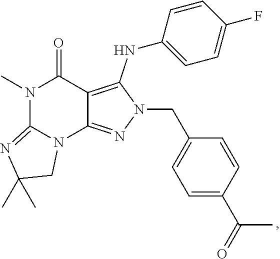 [SMILES: CC(=O)c1ccc(Cn2nc3c(c2Nc2ccc(F)cc2)C(=O)N(C)C2=NC(C)(C)CN23)cc1]

in free or pharmaceutically acceptable salt form.
2.35 Any of the preceding methods, wherein the PDE1 inhibitor is a compound according to:

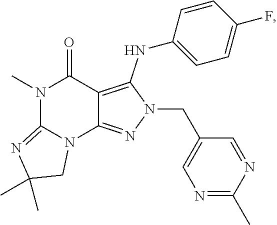 [SMILES: Cc1ncc(Cn2nc3c(c2Nc2ccc(F)cc2)C(=O)N(C)C2=NC(C)(C)CN23)cn1]

in free or pharmaceutically acceptable salt form.
2.36 Any of the preceding methods, wherein the PDE1 inhibitor is a compound according to:

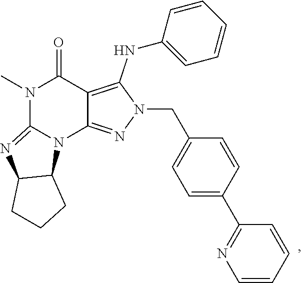 [SMILES: CN1C(=O)c2c(nn(Cc3ccc(-c4ccccn4)cc3)c2Nc2ccccc2)N2C1=N[C@@H]1CCC[C@@H]12]

in free or pharmaceutically acceptable salt form.
2.37 Any of the preceding methods, wherein the PDE1 inhibitor is a compound according to:

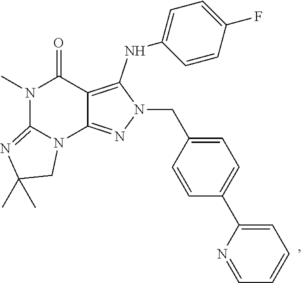 [SMILES: CN1C(=O)c2c(nn(Cc3ccc(-c4ccccn4)cc3)c2Nc2ccc(F)cc2)N2CC(C)(C)N=C12]

in free or pharmaceutically acceptable salt form.
2.38 Any of the preceding methods, wherein the patient is a human.

The disclosure further provides a PDE1 inhibitor for use in a method for treating impaired inhibitory processing, e.g., for use in any of Methods 2, et seq.

The disclosure further provides the use of a PDE1 inhibitor in the manufacture of a medicament for use in a method for treating impaired inhibitory processing, e.g., a medicament for use in any of Methods 2, et seq.

The invention further provides a pharmaceutical composition comprising a PDE1 inhibitor, e.g., any of a Compound of Formulas I, Ia, II, III, IV, V, and/or VI, for use in any of Methods 2, et seq.

Combination Therapies with PDE1 Inhibitors

In some embodiments, the PDE1 inhibitor is administered in combination with other therapeutic modalities. Thus, in addition to the therapies described above, one may also provide to the patient more pharmaceutical therapies for treatment of a condition, disease or disorder associated with the dopamine D1 receptor intracellular pathway, e.g., a condition, disease or disorder characterized by a deficit in attention, cognition, memory and/or inhibitory processing. For example, in addition to the therapies discussed herein, a patient suffering from such a condition, disease or disorder may be administered with a monoamine reuptake inhibitor (e.g. a dopamine reuptake inhibitor, a serotonin reuptake inhibitor, and/or a norepinephrine reuptake inhibitor). A particular form of combination therapy will include the use of PDE1 inhibitors.

Combinations may be achieved by administering a single composition or pharmacological formulation that includes the PDE1 inhibitor and one or more additional therapeutic agents, or by administration of two distinct compositions or formulations, separately, simultaneously or sequentially, wherein one composition includes the PDE1 inhibitor and the other includes the additional therapeutic agent or agents. The therapy using a PDE1 inhibitor may precede or follow administration of the other agent(s) by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In some embodiments, it is contemplated that one would typically contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either a PDE1 inhibitor, or an additional therapeutic agent will be desired. In this regard, various combinations may be employed. By way of illustration, where the PDE1 inhibitor is “A” and the additional therapeutic agent is “B,” the following permutations based on 3 and 4 total administrations are exemplary:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Monoamine reuptake inhibitors as described herein include norepinephrine reuptake inhibitors, serotonin reuptake inhibitors and dopamine reuptake inhibitors, including those having single-, dual- or triple-function for inhibition of one or more of norepinephrine transporter, serotonin transporter and dopamine transporter. Non-limiting examples of monoamine transporters which may be used in the present invention include atomoxetine, reboxetine, nisoxetine, desvenlafaxine, venlafaxine, duloxetine, milnacipran, fluoxetine, sertraline, citalopram, escitalopram, bupropion, and/or nomifensine. Further monoamine transporters are disclosed in International Publication WO 2016/154027 and U.S. Pat. No. 10,188,758, both of which are incorporated by reference in their entirety.

As further examples, dopamine reuptake inhibitors as described herein includes selective or non-selective dopamine reuptake inhibitors. In some embodiments, the dopamine reuptake inhibitors are dual norepinephrine reuptake inhibitors and dopmanine reuptake inhibitors. Non-limiting examples of a dopamine reuptake inhibitor that may be used in the present invention include a 4-Hydroxy-1-methyl-4-(4-methylphenyl)-3-piperidyl-4-methylphenyl ketone, altropane, amfonelic acid, amineptine, BTCP, 3C-PEP, DBL-583, difluoropine, GBR-12783, GBR-12935, GBR-13069, GBR-13098, GYKI-52895, iometopane, methylphenidate, ethylphenidate, modafinil, armodafinil, RTI-229, vanoxerine, adrafinil, amantadine, benztropine, bupropion, fluorenol, medifoxamine, metaphit, rimcazole, venlafaxine, Chaenomeles speciose extract, Oroxylin A, or combinations thereof. Other combinations are likewise contemplated. Some specific agents are described below.

Accordingly, in various embodiments, the present disclosure also provides for a pharmaceutical combination [Combination 1] therapy comprising a pharmaceutically effective amount of a PDE1 inhibitor (e.g., a compound according to Formulas I, II, III, IV, V and/or VI) and a monoamine reuptake inhibitor (e.g., dopamine reuptake inhibitor, serotonin reuptake inhibitor, and/or norepinephrine reuptake inhibitor), for administration to a patient in need thereof in a method for the prophylaxis and/or treatment of a condition, disease or disorder associated with the dopamine D1 receptor intracellular pathway, e.g., a condition, disease or disorder characterized by a deficit in attention, cognition, memory and/or inhibitory processing, e.g., in accordance with any of Method 1, et seq., or for treating impaired inhibitory processing, e.g. in accordance with any of Method 2, et seq.

For example, the present disclosure provides for the following Combinations:
1.1 Combination 1, wherein the patient is suffering from a condition, disease or disorder associated with the dopamine D1 receptor intracellular pathway.
1.2 Any preceding combination, wherein the patient is suffering from condition, disease or disorder selected from an eating disorder (e.g., anorexia nervosa, bulimia nervosa, binge eating disorder, pica, rumination disorder, avoidant/restrictive food intake disorder, purging disorder, night eating disorder, other specified feeding or eating disorder (OSFED)), a substance use disorder (e.g., addiction (e.g., a stimulant addition, such as amphetamine, cocaine, opiate, and/or nicotine), alcoholism), an obsessive-compulsive disorder (e.g., checking, contamination, mental contamination, hoarding, ruminations, intrusive thoughts, symmetry/orderliness), attention deficit hyperactivity disorder (ADHD), premature ejaculation, posttraumatic stress disorder (PTSD), a gambling disorder (e.g., gambling addiction, compulsive-pathological gambling), Tourette's syndrome and/or impulse control and conduct disorders (e.g., oppositional defiant disorder, conduct disorder, intermittent explosive disorder, kleptomania, pyromania).
1.3 Any preceding combination, wherein the patient is suffering from an eating disorder (e.g., anorexia nervosa, bulimia nervosa, binge eating disorder, pica, rumination disorder, avoidant/restrictive food intake disorder, purging disorder, night eating disorder, other specified feeding or eating disorder (OSFED)).
1.4 The preceding combination, wherein the eating disorder is anorexia nervosa, bulimia nervosa, binge eating disorder, pica, rumination disorder, avoidant/restrictive food intake disorder, purging disorder, night eating disorder, other specified feeding and/or eating disorder (OSFED).
1.5 Any preceding combination, wherein the patient is suffering from a substance use disorder (e.g., addiction (e.g., a stimulant addition, such as amphetamine, cocaine, opiate, and/or nicotine), alcoholism).
1.6 The preceding combination, wherein the substance abuse disorder is addiction (e.g., a stimulant addition, such as amphetamine, cocaine, opiate, and/or nicotine) or alcoholism.
1.7 Any preceding combination, wherein the patient is suffering from an obsessive-compulsive disorder (OCD) (e.g., checking OCD, contamination OCD, mental contamination OCD, hoarding OCD, ruminations OCD, intrusive thoughts OCD, symmetry/orderliness OCD).
1.8 The preceding combination, wherein the obsessive-compulsive disorder is checking OCD, contamination OCD, mental contamination OCD, hoarding OCD, ruminations OCD, intrusive thoughts OCD, and/or symmetry/orderliness OCD.
1.9 Any preceding combination, wherein the patient is suffering from attention deficit hyperactivity disorder (ADHD).
1.10 Any preceding combination, wherein the patient is suffering from premature ejaculation.
1.11 Any preceding combination, wherein the patient is suffering from posttraumatic stress disorder (PTSD).
1.12 Any preceding combination, wherein the patient is suffering from a gambling disorder (e.g., gambling addiction, compulsive-pathological gambling).
1.13 The preceding combination, wherein the gambling disorder is gambling addiction or compulsive-pathological gambling.
1.14 Any preceding combination, wherein the condition, disease or disorder is Tourette's syndrome.
1.15 Any preceding combination, wherein the patient is suffering from impulse control and conduct disorders (e.g., oppositional defiant disorder, conduct disorder, intermittent explosive disorder, kleptomania, pyromania).
1.16 The preceding combination, wherein the impulse control and conduct disorder is oppositional defiant disorder, conduct disorder, intermittent explosive disorder, kleptomania and/or pyromania.
1.17 Any of the preceding combinations, wherein the PDE1 inhibitor is administered in an amount of about 0.01 to about 20 mg/kg, e.g. about 0.1 to about 5 mg/kg, e.g. about 1 mg/kg.
1.18 Any of the preceding combinations, wherein the PDE1 inhibitor is administered orally.
1.19 Any of the preceding combinations, wherein the PDE1 inhibitor is administered via injection.
1.20 Any of the preceding combinations, wherein the monoamine reuptake inhibitor is one or more of a dopamine reuptake inhibitor, a serotonin reuptake inhibitor or a norepinephrine reuptake inhibitor.
1.21 Any of the preceding combinations, wherein the monoamine reuptake inhibitor is one or more of 4-Hydroxy-1-methyl-4-(4-methylphenyl)-3-piperidyl-4-methylphenyl ketone, adrafinil, amantadine, atomoxetine, amfonelic acid, amineptine, amitriptyline, amoxapine, benztropine, bupropion, hydroxybupropion, butriptyline, chlorphenamine, citalopram, clomipramine, cocaethylene, desipramine, desmethylcitalopram, desmethylsertraline, desmethylsibutramine, desoxypipradrol, desvenlafaxine, dextroamphetamine, dextromethamphetamine, didesmethylsibutramine, diphenhydramine, dosulepin, doxepin, duloxetine, escitalopram, etoperidone, femoxetine, fluorenol, fluoxetine, fluvoxamine, GBR-12783, GBR-12935, GBR-13069, GBR-13098, GYKI-52895, imipramine, indatraline, iprindole, iometopane, levomilnacipran, lofepramine, maprotiline, mazindol, medifoxamine, metaphit, methylenedioxypyrovalerone (MDPV), methylphenidate, ethylphenidate, mianserin, milnacipran, mirtazapine, modafinil, armodafinil, nefazodone, nefopam, nisoxetine, nomifensine, norfluoxetine, nortriptyline, oxaprotiline, paroxetine, protriptyline, reboxetine, rimcazole, RTI-229, sertraline, sibutramine, trazodone, trimipramine, vanoxerine, venlafaxine, vilazodone, viloxazine, vortioxetine, zimelidine, Chaenorneles speciose extract, and/or Oroxylin A.
1.22 Any of the preceding combinations, wherein the monoamine reuptake inhibitor is one or more of atomoxetine, reboxetine, nisoxetine, desvenlafaxine, venlafaxine, duloxetine, milnacipran, fluoxetine, sertraline, citalopram, escitalopram, bupropion, and/or nomifensine.
1.23 Any of the preceding combinations, wherein the monoamine reuptake inhibitor is a dopamine reuptake inhibitor.
1.24 Any of the preceding combinations, wherein the dopamine reuptake inhibitor is selected from 4-Hydroxy-1-methyl-4-(4-methylphenyl)-3-piperidyl-4-methylphenyl ketone, altropane, amfonelic acid, amineptine, BTCP, 3C-PEP, DBL-583, difluoropine, GBR-12783, GBR-12935, GBR-13069, GBR-13098, GYKI-52895, iometopane, methylphenidate, ethylphenidate, modafinil, armodafinil, RTI-229, vanoxerine, adrafinil, amantadine, benztropine, bupropion, fluorenol, medifoxamine, metaphit, rimcazole, venlafaxine, Chaenomeles speciose extract, Oroxylin A, or combinations thereof.
1.25 Any of the preceding combinations, wherein the monoamine reuptake inhibitor is a norepinephrine and serotonin reuptake inhibitor.
1.26 Any of the preceding combinations, wherein the monoamine reuptake inhibitor is a norepinephrine and serotonin reuptake inhibitor according to Formula VII:

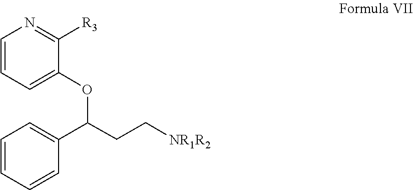

in free or salt form, wherein:
   R₁ and R₂ are independently H or C₁₋₄ alkyl (e.g., methyl or ethyl); and
   R₃ is n-C₂₋₄ alkyl (e.g., ethyl or n-propyl), or —O—C₁₋₄ alkyl (e.g., methoxy
   or ethoxy) optionally substituted hydroxy.
1.27 Any of the preceding combinations, wherein the monoamine reuptake inhibitor is a norepinephrine and serotonin reuptake inhibitor according to

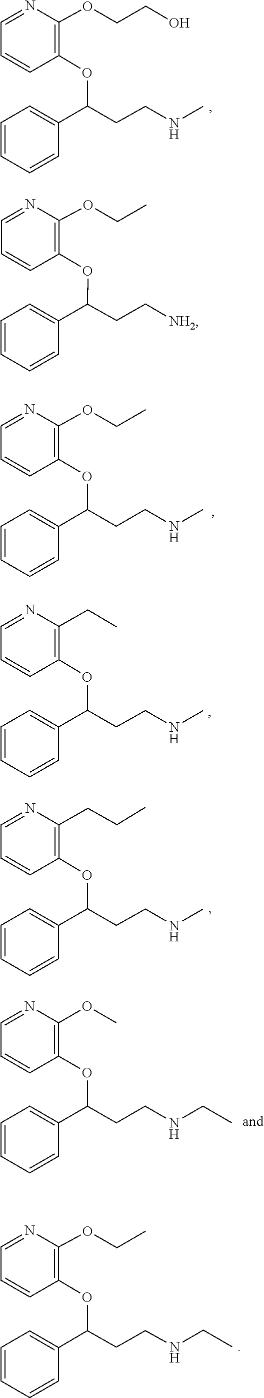

1.28 Any of the preceding combinations, wherein the PDE1 inhibitor is a compound according to any of Formulas I, Ia, II, III, IV, V, and/or VI.
1.29 Any of the preceding combinations, wherein the PDE1 inhibitor is a compound according to Formula Ia.
1.30 Any of the preceding combinations, wherein the PDE1 inhibitor is a compound according to:

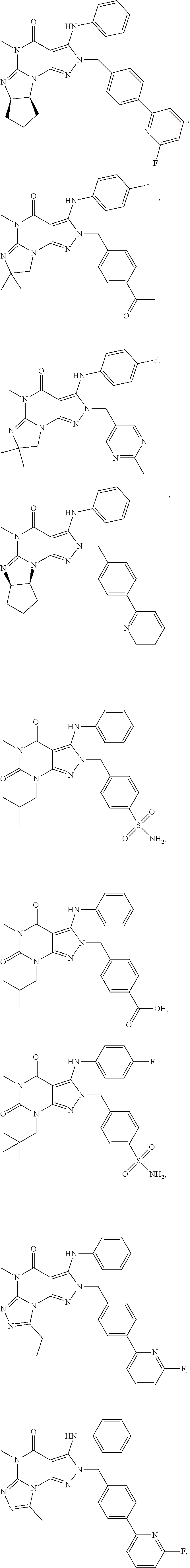

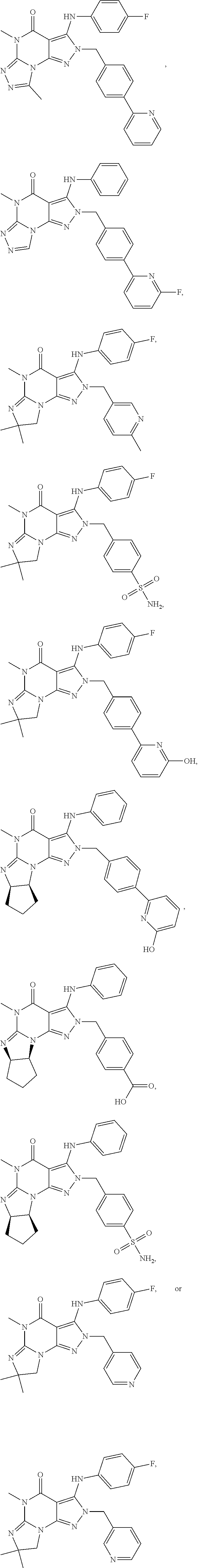

in free or pharmaceutically acceptable salt form.
1.31 Any of the preceding combinations, wherein the PDE1 inhibitor is a compound according to:

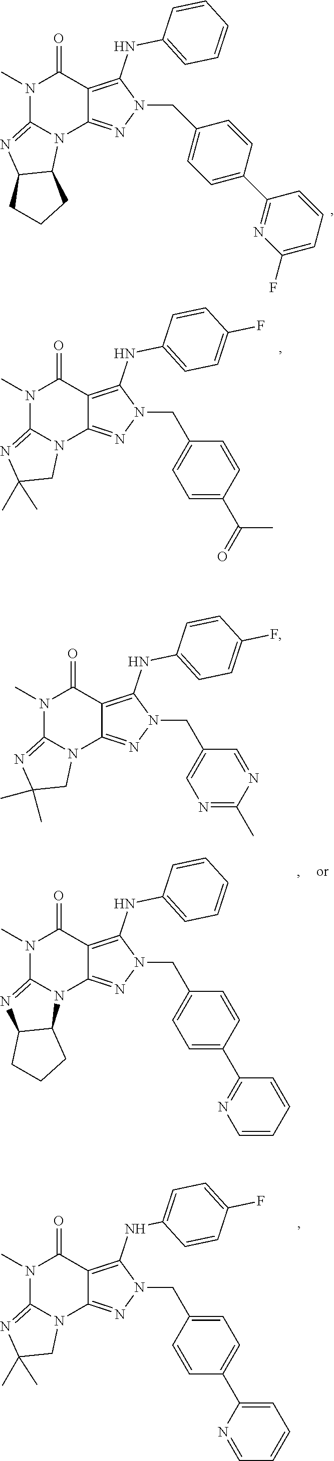

in free or pharmaceutically acceptable salt form.
1.32 Any of the preceding combinations, wherein the PDE1 inhibitor is a compound according to:

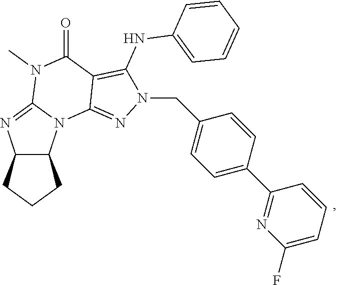 [SMILES: CN1C(=O)c2c(nn(Cc3ccc(-c4cccc(F)n4)cc3)c2Nc2ccccc2)N2C1=N[C@@H]1CCC[C@@H]12]

in free or pharmaceutically acceptable salt form.
1.33 Any of the preceding combinations, wherein the PDE1 inhibitor is a compound according to:

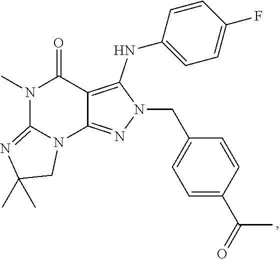 [SMILES: CC(=O)c1ccc(Cn2nc3c(c2Nc2ccc(F)cc2)C(=O)N(C)C2=NC(C)(C)CN23)cc1]

in free or pharmaceutically acceptable salt form.
1.34 Any of the preceding combinations, wherein the PDE1 inhibitor is a compound according to:

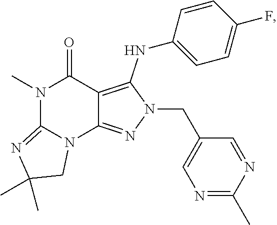 [SMILES: Cc1ncc(Cn2nc3c(c2Nc2ccc(F)cc2)C(=O)N(C)C2=NC(C)(C)CN23)cn1]

in free or pharmaceutically acceptable salt form.
1.35 Any of the preceding combinations, wherein the PDE1 inhibitor is a compound according to:

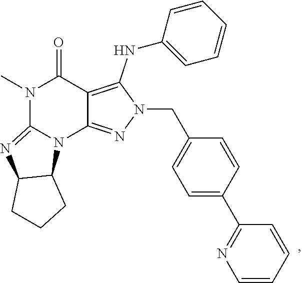 [SMILES: CN1C(=O)c2c(nn(Cc3ccc(-c4ccccn4)cc3)c2Nc2ccccc2)N2C1=N[C@@H]1CCC[C@@H]12]

in free or pharmaceutically acceptable salt form.
1.36 Any of the preceding combinations, wherein the PDE1 inhibitor is a compound according to:

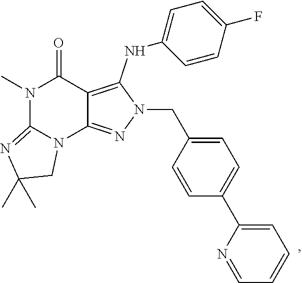 [SMILES: CN1C(=O)c2c(nn(Cc3ccc(-c4ccccn4)cc3)c2Nc2ccc(F)cc2)N2CC(C)(C)N=C12]

in free or pharmaceutically acceptable salt form.
1.37 Any of the preceding combinations, wherein the combination is administered to a human.

### Methods of Making Compounds of the Disclosure

The PDE1 inhibitors of the Disclosure and their pharmaceutically acceptable salts may be made using the methods as described and exemplified in U.S. Pat. No. 8,273,750, US 2006/0173878, U.S. Pat. No. 8,273,751, US 2010/0273753, U.S. Pat. Nos. 8,697,710, 8,664,207, 8,633,180, 8,536,159, US 2012/0136013, US 2011/0281832, US 2013/0085123, US 2013/0324565, US 2013/0338124, US 2013/0331363, WO 2012/171016, and WO 2013/192556, and by methods similar thereto and by methods known in the chemical art. Such methods include, but not limited to, those described below. If not commercially available, starting materials for these processes may be made by procedures, which are selected from the chemical art using techniques which are similar or analogous to the synthesis of known compounds.

Various PDE1 inhibitors and starting materials therefor may be prepared using methods described in US 2008-0188492 A1, US 2010-0173878 A1, US 2010-0273754 A1, US 2010-0273753 A1, WO 2010/065153, WO 2010/065151, WO 2010/065151, WO 2010/065149, WO 2010/065147, WO 2010/065152, WO 2011/153129, WO 2011/133224, WO 2011/153135, WO 2011/153136, WO 2011/153138. All references cited herein are hereby incorporated by reference in their entirety.

Further PDE1 inhibitors and related methods are disclosed in U.S. Provisional Application 62/833,481, which is hereby incorporated by reference in its entirety. Additional related PDE1 inhibitors and related methods are disclosed in International Publication WO2018/049417, which is hereby incorporated by reference in its entirety.

The Compounds of the Disclosure include their enantiomers, diastereomers and racemates, as well as their polymorphs, hydrates, solvates and complexes. Some individual compounds within the scope of this disclosure may contain double bonds. Representations of double bonds in this disclosure are meant to include both the E and the Z isomer of the double bond. In addition, some compounds within the scope of this disclosure may contain one or more asymmetric centers. This disclosure includes the use of any of the optically pure stereoisomers as well as any combination of stereoisomers.

It is also intended that the Compounds of the Disclosure encompass their stable and unstable isotopes. Stable isotopes are nonradioactive isotopes which contain one additional neutron compared to the abundant nuclides of the same species (i.e., element). It is expected that the activity of compounds comprising such isotopes would be retained, and such compound would also have utility for measuring pharmacokinetics of the non-isotopic analogs. For example, the hydrogen atom at a certain position on the Compounds of the Disclosure may be replaced with deuterium (a stable isotope which is non-radioactive). Examples of known stable isotopes include, but not limited to, deuterium, ¹³C, ¹⁵N, ¹⁸O. Alternatively, unstable isotopes, which are radioactive isotopes which contain additional neutrons compared to the abundant nuclides of the same species (i.e., element), e.g., ¹²³I, ¹³¹I, ¹²⁵I, ¹¹C, ¹⁸F, may replace the corresponding abundant species of I, C and F. Another example of useful isotope of the compound of the disclosure is the ¹¹C isotope. These radio isotopes are useful for radio-imaging and/or pharmacokinetic studies of the compounds of the disclosure.

Melting points are uncorrected and (dec) indicates decomposition. Temperature are given in degrees Celsius (° C.); unless otherwise stated, operations are carried out at room or ambient temperature, that is, at a temperature in the range of 18-25° C. Chromatography means flash chromatography on silica gel; thin layer chromatography (TLC) is carried out on silica gel plates. NMR data is in the delta values of major diagnostic protons, given in parts per million (ppm) relative to tetramethylsilane (TMS) as an internal standard. Conventional abbreviations for signal shape are used. Coupling constants (J) are given in Hz. For mass spectra (MS), the lowest mass major ion is reported for molecules where isotope splitting results in multiple mass spectral peaks Solvent mixture compositions are given as volume percentages or volume ratios. In cases where the NMR spectra are complex, only diagnostic signals are reported.

The words “treatment” and “treating” are to be understood accordingly as embracing treatment or amelioration of symptoms of disease as well as treatment of the cause of the disease.

For methods of treatment, the word “effective amount” is intended to encompass a therapeutically effective amount to treat a specific disease or disorder.

The term “patient” includes human or non-human (i.e., animal) patient. In particular embodiment, the disclosure encompasses both human and nonhuman. In another embodiment, the disclosure encompasses nonhuman. In other embodiment, the term encompasses human.

The term “comprising” as used in this disclosure is intended to be open-ended and does not exclude additional, un-recited elements or method steps.

Dosages employed in practicing the present disclosure will of course vary depending, e.g. on the particular disease or condition to be treated, the particular Compounds of the Disclosure used, the mode of administration, and the therapy desired. Compounds of the Disclosure may be administered by any suitable route, including orally, parenterally, transdermally, or by inhalation, but are preferably administered orally. In general, satisfactory results, e.g. for the treatment of diseases as hereinbefore set forth are indicated to be obtained on oral administration at dosages of the order from about 0.01 to 2.0 mg/kg. In larger mammals, for example humans, an indicated daily dosage for oral administration of both the PDE1 inhibitor will accordingly be in the range of from about 0.50 to 300 mg, conveniently administered once, or in divided doses 2 to 4 times, daily or in sustained release form. Unit dosage forms for oral administration thus for example may comprise from about 0.2 to 150 or 300 mg, e.g. from about 0.2 or 2.0 to 10, 25, 50, 75 100, 150, or 200 mg of a Compound of the Disclosure, together with a pharmaceutically acceptable diluent or carrier therefor.

Compounds of the Disclosure, particularly for use or administration in any of Methods 1 or 2, et seq., may be administered at higher doses as necessary. It is envisioned that administration of a PDE1 inhibitor for such a method may be in the range of about 50 mg to 1000 mg daily. For example, a patient being administered a PDE1 inhibitor for a condition according to any of Methods 1-6, et seq., may be administered a PDE1 inhibitor according to Formulas I, Ia, II, III, IV, V, and/or VI in an amount of 50 mg to 1000 mg daily, 50 mg to 900 mg daily, 50 mg to 800 mg daily, 50 mg to 700 mg daily, 50 mg to 600 mg daily, 50 mg to 500 mg daily, 50 mg to 400 mg daily, 50 mg to 350 mg daily, 50 mg to 300 mg daily, 50 mg to 250 mg daily, 50 mg to 200 mg daily, 50 mg to 150 mg daily or 50 mg to 100 mg daily.

Compounds of the Disclosure may be administered by any satisfactory route, including orally, parenterally (intravenously, intramuscular or subcutaneous) or transdermally, but are preferably administered orally. In certain embodiments, the Compounds of the Disclosure, e.g., in depot formulation, is preferably administered parenterally, e.g., by injection.

The Compounds of the Disclosure and the Pharmaceutical Compositions of the Disclosure of the Disclosure may be used in combination with one or more additional therapeutic agents, particularly at lower dosages than when the individual agents are used as a monotherapy so as to enhance the therapeutic activities of the combined agents without causing the undesirable side effects commonly occur in conventional monotherapy. Therefore, the Compounds of the Disclosure may be simultaneously, separately, sequentially, or contemporaneously administered with other agents useful in treating disease. In another example, side effects may be reduced or minimized by administering a Compound of the Disclosure in combination with one or more additional therapeutic agents in free or salt form, wherein the dosages of (i) the second therapeutic agent(s) or (ii) both Compound of the Disclosure and the second therapeutic agent, are lower than if the agent/compound are administered as a monotherapy. By way of non-limiting example, such additional therapeutic agents may include ACE inhibitors, Angiotensin II receptor antagonists, calcium channel blockers, etc.

The term “simultaneously” when referring to a therapeutic use means administration of two or more active ingredients at or about the same time by the same route of administration.

The term “separately” when referring to a therapeutic use means administration of two or more active ingredients at or about the same time by different route of administration.

Pharmaceutical compositions comprising Compounds of the Disclosure may be prepared using conventional diluents or excipients and techniques known in the galenic art. Thus, oral dosage forms may include tablets, capsules, solutions, suspensions and the like.

### EXAMPLES

### Example 1: Determining the Engagement of PDE1 Inhibitors in the Human Brain

A randomized, double-blind, placebo-controlled, within-subjects study was carried out with healthy human individuals to determine whether the PDE1 inhibitors according to the present disclosure induce changes in blood oxygen level dependent functional magnetic resonance imaging (BOLD-fMRI) signals in the dorsal anterior insula (dAI) during the extinction phase of a fear conditioning task. To test these effects, Compound 1 was administered to healthy human patients. Compound 1 (ITI-214) is shown below:

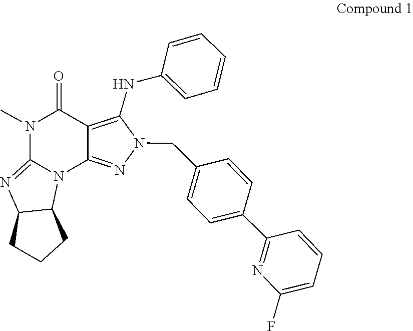 [SMILES: CN1C(=O)c2c(nn(Cc3ccc(-c4cccc(F)n4)cc3)c2Nc2ccccc2)N2C1=N[C@@H]1CCC[C@@H]12]

The insula is a small brain region that is essential for the mind-body connection, where body sensations are translated into emotion. Anterior insula reactivity has been associated with anticipating significant events, and increased anterior insula activity has been associated with anxiety. Conditioning, including fear conditioning and extinction, is a brain activity during learning. It has previously been shown that the PDE1 inhibitors as disclosed herein enhances dopamine and enhances learning in animal models. To test this hypothesis, a neutral abstract image (CSplus) is paired repeatedly with a scream. The task consisted of three components: a brief familiarization period, fear acquisition, and fear extinction. A neutral stimulus (CSminus) never paired with an unconditioned stimulus, is used as a control.

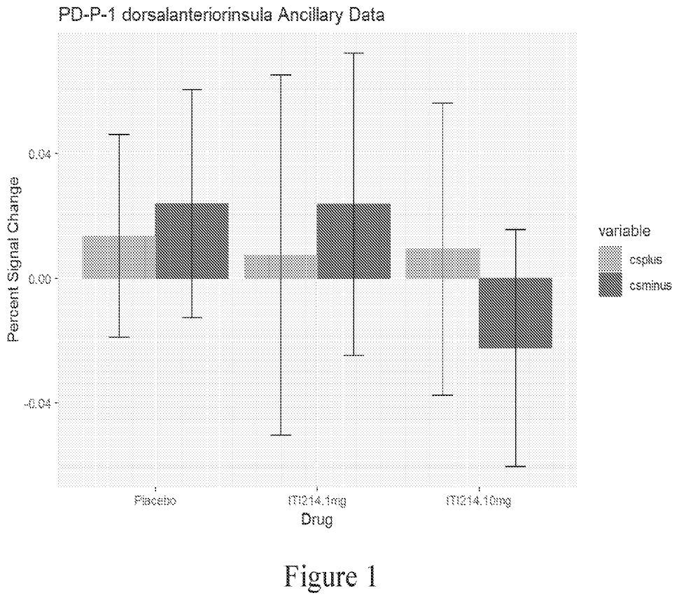

Patients were administered single oral doses of placebo, or doses of Compound 1 at 1 mg or 10 mg. Brain activity was then measured during BOLD-fMRI scanning at 3 Tesla. All fMRI analyses were conducted in AFNI. Region-of-interest (ROI) analyses for the dAI, IFG, and other prespecified regions were computed using linear mixed models in R. The brain's response is measured in each subject by fMRI scans, which is summarized in FIG. 1. As shown, administration of Compound 1 slightly attenuated the insula activity during fear extinction in this study.

Additional tests were carried out to determine whether the PDE1 inhibitors according to the present disclosure induce changes in BOLD-fMRI signals in the inferior frontal gyms (IFG) during a stop signal task. The inferior frontal gyms is a brain region important for inhibition of response or the ability to refrain from performing a response after given a signal to stop. The Stop Signal task is a way to measure behavioral and neural responses to inhibitory processing.

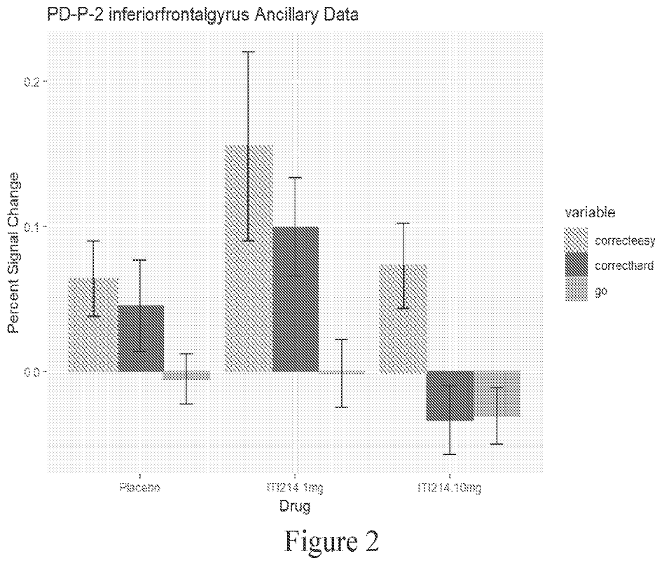

Patients were again administered single oral doses of placebo, Compound 1 at 1 mg or 10 mg. Brain activity was then measured during BOLD-fMRI scanning at 3 Tesla. In this study, participants are presented with a visual and/or audio cue and two buttons. The subjects are instructed to press the left button as quickly as possible when an ‘X’ appears on a screen and the right button when an ‘O’ appears. Participants also are instructed not to press either button when they hear a tone, which constitute the Stop trials. There are two different kinds of Stop trials presented during the task, namely easy and hard trials. Easy trials consist of the tone cue being presented simulataneously with the visual ‘X’ or ‘O’ making it easier to suppress a response. Hard trials consist of the tone being presented after a brief delay, making it harder to process the tone and suppress a response. Brain activity in the IFG was measured in each subject by fMRI scans, and is summarized in FIG. 2. As shown, administration of Compound 1 (1 mg) significantly increased activation in the inferior frontal gyrus during correctly inhibited Stop trials during both easy trials (correcteasy) and hard trials (correcthard).

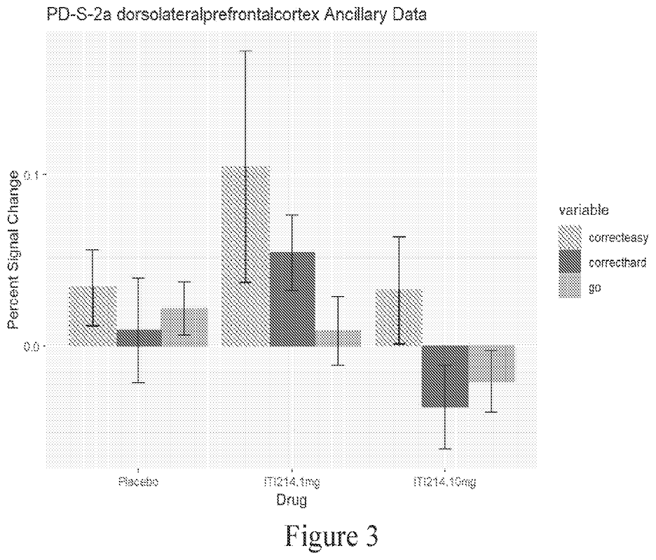

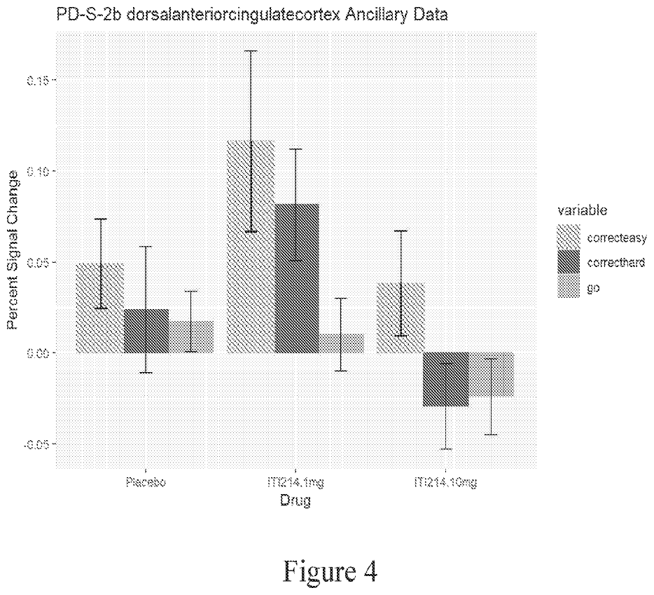

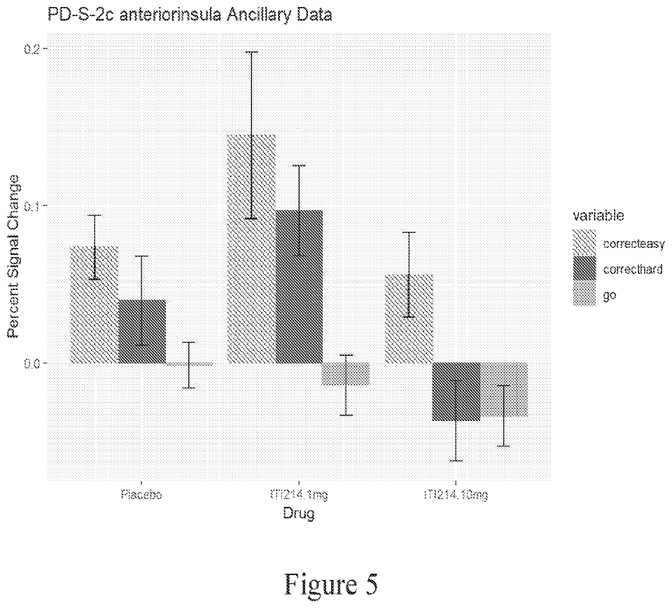

Similar patterns of increased activity were observed in the dorsolateral prefrontal cortex (FIG. 3), the dorsal anterior cingulate cortex (FIG. 4), and the anterior insula (FIG. 5) as the activity observed in the inferior frontal gyrus. This consistency provides confidence in the findings.

Compound 1 improved cognitive performance without psychomotor hyperactivity in preclinical models, and was predicted to improve cognitive performance in humans and increase activation of the frontal gyms. Compound 1 (1 mg) also increased activation in the frontal gyrus, consistent with improving cognition in disorders such as ADHD and improving inhibitory processes in disorders such as PTSD. These results provide evidence of central nervous system engagement and enhanced cognitive processing consistent with the mechanism of action of Compound 1 and indicate potential utility for treatment of an array of disorders associated with impaired inhibitory processing.

Alternative combinations and variations of the examples provided will become apparent based on the disclosure. It is not possible to provide specific examples for all of the many possible variations of the embodiments described, but such combinations and variations may be claims that eventually issue.

1. A method for prophylaxis and/or treatment of a condition, disease or disorder associated with the dopamine D1 receptor intracellular pathway, e.g., a condition, disease or disorder characterized by a deficit in attention, cognition, memory and/or inhibitory processing, the method comprising administering a pharmaceutically effective amount of a PDE1 inhibitor (i.e., a compound according to Formulas I, Ia, II, III, IV, V, and/or VI) to a subject in need thereof.

2. The method according to claim 1, wherein the condition, disease or disorder is characterized by a deficit in attention, cognition, memory and/or inhibitory processing.

3. The method according to claim 1, wherein the condition, disease or disorder is selected from an eating disorder (e.g., anorexia nervosa, bulimia nervosa, binge eating disorder, pica, rumination disorder, avoidant/restrictive food intake disorder, purging disorder, night eating disorder, other specified feeding or eating disorder (OSFED)), a substance use disorder (e.g., addiction (e.g., a stimulant addition, such as amphetamine, cocaine, opiate, and/or nicotine), alcoholism), an obsessive-compulsive disorder (e.g., checking, contamination, mental contamination, hoarding, ruminations, intrusive thoughts, symmetry/orderliness), attention deficit hyperactivity disorder (ADHD), premature ejaculation, posttraumatic stress disorder (PTSD), a gambling disorder (e.g., gambling addiction, compulsive-pathological gambling), Tourette's syndrome and/or impulse control and conduct disorders (e.g., oppositional defiant disorder, conduct disorder, intermittent explosive disorder, kleptomania, pyromania).

4. A method of treatment for impaired inhibitory processing, the method comprising administering a pharmaceutically effective amount of a PDE1 inhibitor (i.e., a compound according to Formulas I, Ia, II, III, IV, V, and/or VI) to a patient in need thereof.

5. The method according to claim 4, wherein the patient is suffering from a condition, disease or disorder associated with the dopamine D1 receptor intracellular pathway.

6. The method according to claim 4, wherein the patient is suffering from condition, disease or disorder selected from an eating disorder (e.g., anorexia nervosa, bulimia nervosa, binge eating disorder, pica, rumination disorder, avoidant/restrictive food intake disorder, purging disorder, night eating disorder, other specified feeding or eating disorder (OSFED)), a substance use disorder (e.g., addiction (e.g., a stimulant addition, such as amphetamine, cocaine, opiate, and/or nicotine), alcoholism), an obsessive-compulsive disorder (e.g., checking, contamination, mental contamination, hoarding, ruminations, intrusive thoughts, symmetry/orderliness), attention deficit hyperactivity disorder (ADHD), premature ejaculation, posttraumatic stress disorder (PTSD), a gambling disorder (e.g., gambling addiction, compulsive-pathological gambling), Tourette's syndrome and/or impulse control and conduct disorders (e.g., oppositional defiant disorder, conduct disorder, intermittent explosive disorder, kleptomania, pyromania).

7. The method according to claim 1, wherein the PDE1 inhibitor is a compound selected from: 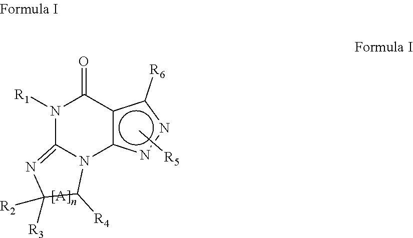
wherein
(i) R₁ is H or C₁₋₄ alkyl (e.g., methyl);
(ii) R₄ is H or C₁₋₄ alkyl and R₂ and R₃ are, independently, H or C₁₋₄ alkyl (e.g., R₂ and R₃ are both methyl, or R₂ is H and R₃ is isopropyl), aryl, heteroaryl, (optionally hetero)arylalkoxy, or (optionally hetero)arylalkyl; or
R₂ is H and R₃ and R₄ together form a di-, tri- or tetramethylene bridge (pref. wherein the R₃ and R₄ together have the cis configuration, e.g., where the carbons carrying R₃ and R₄ have the R and S configurations, respectively);
(iii) R₅ is a substituted heteroarylalkyl, e.g., substituted with haloalkyl;
or R₅ is attached to one of the nitrogens on the pyrazolo portion of Formula I and is a moiety of Formula A 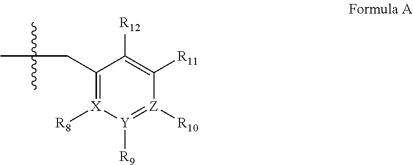
wherein X, Y and Z are, independently, N or C, and R₈, R₉, R₁₁ and Rig are independently H or halogen (e.g., Cl or F), and R₁₀ is halogen, alkyl, cycloalkyl, haloalkyl (e.g., trifluoromethyl), aryl (e.g., phenyl), heteroaryl (e.g., pyridyl (for example pyrid-2-yl) optionally substituted with halogen, or thiadiazolyl (e.g., 1,2,3-thiadiazol-4-yl)), diazolyl, triazolyl, tetrazolyl, arylcarbonyl (e.g., benzoyl), alkylsulfonyl (e.g., methylsulfonyl), heteroarylcarbonyl, or alkoxycarbonyl; provided that when X, Y, or Z is nitrogen, R₈, R₉, or R₁₀, respectively, is not present; and
(iv) R₆ is H, alkyl, aryl, heteroaryl, arylalkyl (e.g., benzyl), arylamino (e.g., phenylamino), heteroarylamino, N,N-dialkylamino, N,N-diarylamino, or N-aryl-N-(arylalkyl)amino (e.g., N-phenyl-N-(1,1′-biphen-4-ylmethyl)amino); and
(v) n=0 or 1;
(vi) when n=1, A is —C(R₁₃R₁₄)—
wherein R₁₃ and R₁₄, are, independently, H or C₁₋₄ alkyl, aryl, heteroaryl, (optionally hetero)arylalkoxy or (optionally hetero)arylalkyl;
in free, salt or prodrug form, including its enantiomers, diastereoisomers and racemates; 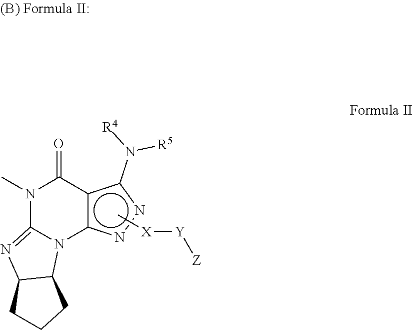
(i) X is C₁₋₆ alkylene (e.g., methylene, ethylene or prop-2-yn-1-ylene);
(ii) Y is a single bond, alkynylene (e.g., —C≡C—), arylene (e.g., phenylene) or heteroarylene (e.g., pyridylene);
(iii) Z is H, aryl (e.g., phenyl), heteroaryl (e.g., pyridyl, e.g., pyrid-2-yl), halo (e.g., F, Br, Cl), haloC₁₋₆ alkyl (e.g., trifluoromethyl), —C(O)—R₁, —N(R₂)(R₃), or C₃₋₇ cycloalkyl optionally containing at least one atom selected from a group consisting of N or 0 (e.g., cyclopentyl, cyclohexyl, tetrahydro-2H-pyran-4-yl, or morpholinyl);
(iv) R₁ is C₁₋₆ alkyl, haloC₁₋₆ alkyl, —OH or —OC₁₋₆ alkyl (e.g., —OCH₃);
(v) R₂ and R₃ are independently H or C₁₋₆ alkyl;
(vi) R₄ and R₅ are independently H, C₁₋₆ alky or aryl (e.g., phenyl) optionally substituted with one or more halo (e.g., fluorophenyl, e.g., 4-fluorophenyl), hydroxy (e.g., hydroxyphenyl, e.g., 4-hydroxyphenyl or 2-hydroxyphenyl) or C₁₋₆ alkoxy;
(vii) wherein X, Y and Z are independently and optionally substituted with one or more halo (e.g., F, Cl or Br), C₁₋₆ alkyl (e.g., methyl), haloC₁₋₆ alkyl (e.g., trifluoromethyl), for example, Z is heteroaryl, e.g., pyridyl substituted with one or more halo (e.g., 6-fluoropyrid-2-yl, 5-fluoropyrid-2-yl, 6-fluoropyrid-2-yl, 3-fluoropyrid-2-yl, 4-fluoropyrid-2-yl, 4,6-dichloropyrid-2-yl), haloC₁₋₆ alkyl (e.g., 5-trifluoromethylpyrid-2-yl) or C₁₋₆-alkyl (e.g., 5-methylpyrid-2-yl), or Z is aryl, e.g., phenyl, substituted with one or more halo (e.g., 4-fluorophenyl),
in free, salt or prodrug form; 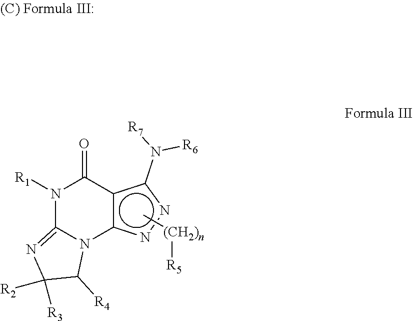
wherein
(i) R₁ is H or C₁₋₄ alkyl (e.g., methyl or ethyl);
(ii) R₂ and R₃ are independently H or C₁₋₆ alkyl (e.g., methyl or ethyl);
(iii) R₄ is H or C₁₋₄ alkyl (e.g., methyl or ethyl);
(iv) R₅ is aryl (e.g., phenyl) optionally substituted with one or more groups independently selected from —C(═O)—C₁₋₆ alkyl (e.g., —C(═O)—CH₃) and C₁₋₆-hydroxyalkyl (e.g., 1-hydroxyethyl);
(v) R₆ and R₇ are independently H or aryl (e.g., phenyl) optionally substituted with one or more groups independently selected from C₁₋₆ alkyl (e.g., methyl or ethyl) and halogen (e.g., F or Cl), for example unsubstituted phenyl or phenyl substituted with one or more halogen (e.g., F) or phenyl substituted with one or more C₁₋₆ alkyl and one or more halogen or phenyl substituted with one C₁₋₆ alkyl and one halogen, for example 4-fluorophenyl or 3,4-difluorophenyl or 4-fluoro-3-methylphenyl; and
(vi) n is 1, 2, 3, or 4,
in free or salt form; 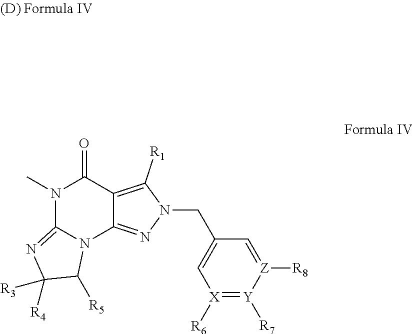
in free or salt form, wherein
(i) R₁ is C₁₋₄ alkyl (e.g., methyl or ethyl), or —NH(R₂), wherein R₂ is phenyl optionally substituted with halo (e.g., fluoro), for example, 4-fluorophenyl;
(ii) X, Y and Z are, independently, N or C;
(iii) R₃, R₄ and R₅ are independently H or C₁₋₄ alkyl (e.g., methyl); or R₃ is H and R₄ and R₅ together form a tri-methylene bridge (pref. wherein the R₄ and R₅ together have the cis configuration, e.g., where the carbons carrying R₄ and R₅ have the R and S configurations, respectively),
(iv) R₆, R₇ and R₈ are independently:
H,
pyrid-2-yl substituted with hydroxy, or
—S(O)₂—NH₂;
provided that when X, Y and/or Z are N, then R₆, R₇ and/or R₈, respectively, are not present; and
when X, Y and Z are all C, then at least one of R₆, R₇ or R₈ is —S(O)₂—NH₂ or pyrid-2-yl substituted with hydroxy,
in free or salt form; 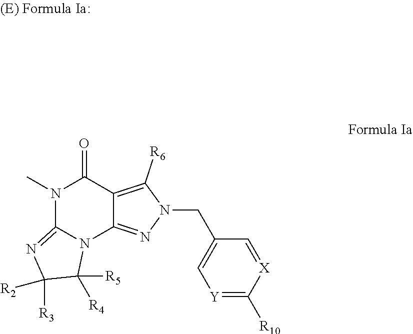
wherein
(i) R₂ and R₅ are independently H or hydroxy and R₃ and R₄ together form a tri- or tetra-methylene bridge [pref. with the carbons carrying R₃ and R₄ having the R and S configuration respectively]; or R₂ and R₃ are each methyl and R₄ and R₅ are each H; or R₂, R₄ and R₅ are H and R₃ is isopropyl [pref. the carbon carrying R₃ having the R configuration];
(ii) R₆ is (optionally halo-substituted) phenylamino, (optionally halo-substituted) benzylamino, C₁₋₄alkyl, or C₁₋₄alkyl sulfide; for example, phenylamino or 4-fluorophenylamino;
(iii) R₁₀ is C₁₋₄alkyl, methylcarbonyl, hydroxyethyl, carboxylic acid, sulfonamide, (optionally halo- or hydroxy-substituted) phenyl, (optionally halo- or hydroxy-substituted) pyridyl (for example 6-fluoropyrid-2-yl), or thiadiazolyl (e.g., 1,2,3-thiadiazol-4-yl); and
X and Y are independently C or N,
in free, pharmaceutically acceptable salt or prodrug form, including its enantiomers, diastereoisomers and racemates; 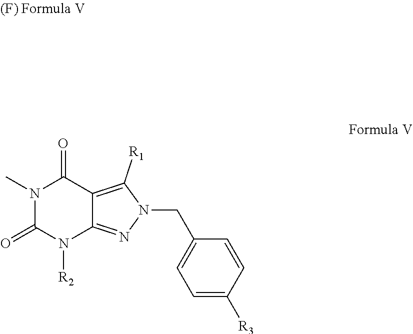
wherein
(i) R₁ is —NH(R₄), wherein R₄ is phenyl optionally substituted with halo (e.g., fluoro), for example, 4-fluorophenyl;
(ii) R₂ is H or C₁₋₆ alkyl (e.g., methyl, isobutyl or neopentyl);
(iii) R₃ is —SO₂NH₂ or —COOH;
in free or salt form; and/or 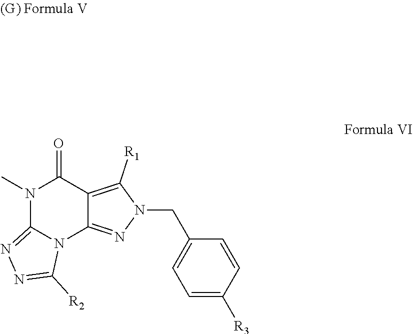
(i) R₁ is —NH(R₄), wherein R₄ is phenyl optionally substituted with halo (e.g., fluoro), for example, 4-fluorophenyl;
(ii) R₂ is H or C₁₋₆ alkyl (e.g., methyl or ethyl);
(iii) R₃ is H, halogen (e.g., bromo), C₁₋₆ alkyl (e.g., methyl), aryl optionally substituted with halogen (e.g., 4-fluorophenyl), heteroaryl optionally substituted with halogen (e.g., 6-fluoropyrid-2-yl or pyrid-2-yl), or acyl (e.g., acetyl),
in free or salt form.

8. The method according to claim 1, wherein the PDE1 inhibitor is selected from any of the following: 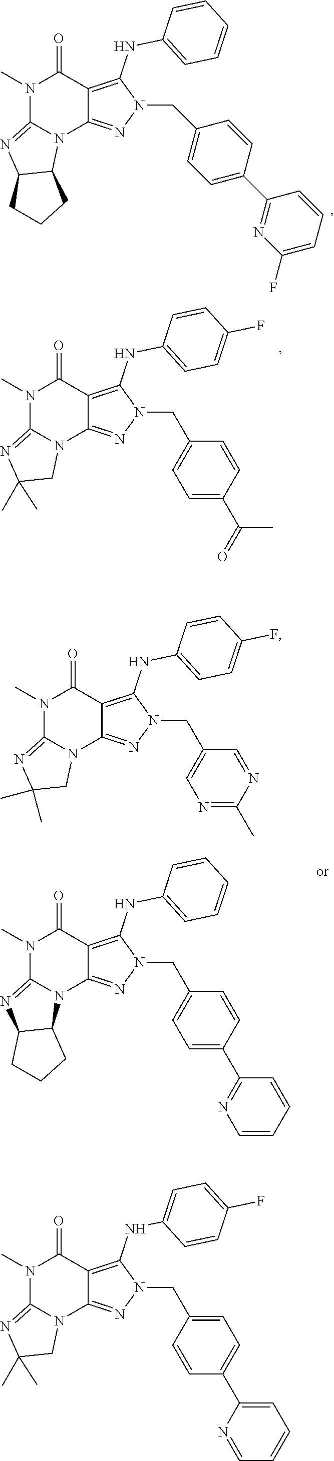
in free or pharmaceutically acceptable salt form.

9. The method according to claim 1, wherein the PDE1 inhibitor is administered in combination with a monoamine reuptake inhibitor (e.g., a dopamine reuptake inhibitor, a serotonin reuptake inhibitor, and/or an epinephrine reuptake inhibitor).

10. A pharmaceutical combination therapy comprising a pharmaceutically effective amount of a PDE1 inhibitor and a monoamine reuptake inhibitor (e.g., a dopamine reuptake inhibitor, a serotonin reuptake inhibitor, and/or an epinephrine reuptake inhibitor), for administration in a method according to claim 1.

11. The method according to claim 4, wherein the PDE1 inhibitor is a compound selected from: 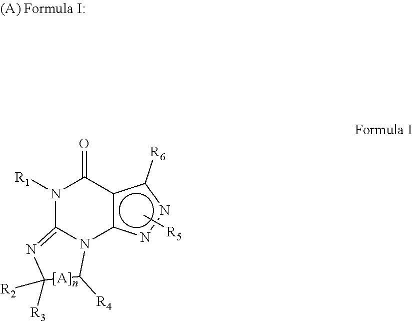
wherein
(i) R₁ is H or C₁₋₄ alkyl (e.g., methyl);
(ii) R₄ is H or C₁₋₄ alkyl and R₂ and R₃ are, independently, H or C₁₋₄ alkyl (e.g., R₂ and R₃ are both methyl, or R₂ is H and R₃ is isopropyl), aryl, heteroaryl, (optionally hetero)arylalkoxy, or (optionally hetero)arylalkyl; or
R₂ is H and R₃ and R₄ together form a di-, tri- or tetramethylene bridge (pref. wherein the R₃ and R₄ together have the cis configuration, e.g., where the carbons carrying R₃ and R₄ have the R and S configurations, respectively);
(iii) R₅ is a substituted heteroarylalkyl, e.g., substituted with haloalkyl;
or R₅ is attached to one of the nitrogens on the pyrazolo portion of Formula I and is a moiety of Formula A 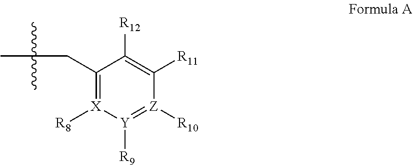
wherein X, Y and Z are, independently, N or C, and R₈, R₉, R₁₁ and R₁₂ are independently H or halogen (e.g., Cl or F), and R₁₀ is halogen, alkyl, cycloalkyl, haloalkyl (e.g., trifluoromethyl), aryl (e.g., phenyl), heteroaryl (e.g., pyridyl (for example pyrid-2-yl) optionally substituted with halogen, or thiadiazolyl (e.g., 1,2,3-thiadiazol-4-yl)), diazolyl, triazolyl, tetrazolyl, arylcarbonyl (e.g., benzoyl), alkylsulfonyl (e.g., methylsulfonyl), heteroarylcarbonyl, or alkoxycarbonyl; provided that when X, Y, or Z is nitrogen, R₈, R₉, or R₁₀, respectively, is not present; and
(iv) R₆ is H, alkyl, aryl, heteroaryl, arylalkyl (e.g., benzyl), arylamino (e.g., phenylamino), heteroarylamino, N,N-dialkylamino, N,N-diarylamino, or N-aryl-N-(arylalkyl)amino (e.g., N-phenyl-N-(1,1′-biphen-4-ylmethyl)amino); and
(v) n=0 or 1;
(vi) when n=1, A is —C(R₁₃R₁₄)—
wherein R₁₃ and R₁₄, are, independently, H or C₁₋₄ alkyl, aryl, heteroaryl, (optionally hetero)arylalkoxy or (optionally hetero)arylalkyl;
in free, salt or prodrug form, including its enantiomers, diastereoisomers and racemates; 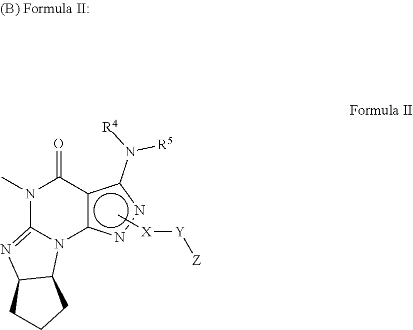
(i) X is C₁₋₆ alkylene (e.g., methylene, ethylene or prop-2-yn-1-ylene);
(ii) Y is a single bond, alkynylene (e.g., —C≡C—), arylene (e.g., phenylene) or heteroarylene (e.g., pyridylene);
(iii) Z is H, aryl (e.g., phenyl), heteroaryl (e.g., pyridyl, e.g., pyrid-2-yl), halo (e.g., F, Br, Cl), haloC₁₋₆ alkyl (e.g., trifluoromethyl), —C(O)—R₁, —N(R₂)(R₃), or C₃₋₇ cycloalkyl optionally containing at least one atom selected from a group consisting of N or 0 (e.g., cyclopentyl, cyclohexyl, tetrahydro-2H-pyran-4-yl, or morpholinyl);
(iv) R₁ is C₁₋₆ alkyl, haloC₁₋₆ alkyl, —OH or —OC₁₋₆ alkyl (e.g., —OCH₃);
(v) R₂ and R₃ are independently H or C₁₋₆ alkyl;
(vi) R₄ and R₅ are independently H, C₁₋₆ alky or aryl (e.g., phenyl) optionally substituted with one or more halo (e.g., fluorophenyl, e.g., 4-fluorophenyl), hydroxy (e.g., hydroxyphenyl, e.g., 4-hydroxyphenyl or 2-hydroxyphenyl) or C₁₋₆ alkoxy;
(vii) wherein X, Y and Z are independently and optionally substituted with one or more halo (e.g., F, Cl or Br), C₁₋₆ alkyl (e.g., methyl), haloC₁₋₆ alkyl (e.g., trifluoromethyl), for example, Z is heteroaryl, e.g., pyridyl substituted with one or more halo (e.g., 6-fluoropyrid-2-yl, 5-fluoropyrid-2-yl, 6-fluoropyrid-2-yl, 3-fluoropyrid-2-yl, 4-fluoropyrid-2-yl, 4,6-dichloropyrid-2-yl), haloC₁₋₆ alkyl (e.g., 5-trifluoromethylpyrid-2-yl) or C₁₋₆-alkyl (e.g., 5-methylpyrid-2-yl), or Z is aryl, e.g., phenyl, substituted with one or more halo (e.g., 4-fluorophenyl),
in free, salt or prodrug form; 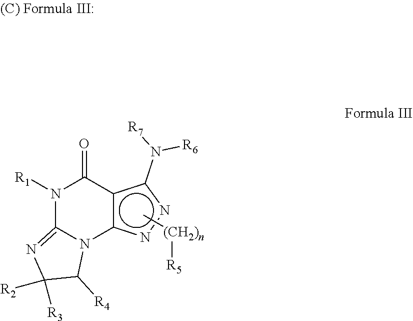
wherein
(i) R₁ is H or C₁₋₄ alkyl (e.g., methyl or ethyl);
(ii) R₂ and R₃ are independently H or C₁₋₆ alkyl (e.g., methyl or ethyl);
(iii) R₄ is H or C₁₋₄ alkyl (e.g., methyl or ethyl);
(iv) R₅ is aryl (e.g., phenyl) optionally substituted with one or more groups independently selected from —C(═O)—C₁₋₆ alkyl (e.g., —C(═O)—CH₃) and C₁₋₆-hydroxyalkyl (e.g., 1-hydroxyethyl);
(v) R₆ and R₇ are independently H or aryl (e.g., phenyl) optionally substituted with one or more groups independently selected from C₁₋₆ alkyl (e.g., methyl or ethyl) and halogen (e.g., F or Cl), for example unsubstituted phenyl or phenyl substituted with one or more halogen (e.g., F) or phenyl substituted with one or more C₁₋₆ alkyl and one or more halogen or phenyl substituted with one C₁₋₆ alkyl and one halogen, for example 4-fluorophenyl or 3,4-difluorophenyl or 4-fluoro-3-methylphenyl; and
(vi) n is 1, 2, 3, or 4,
in free or salt form; 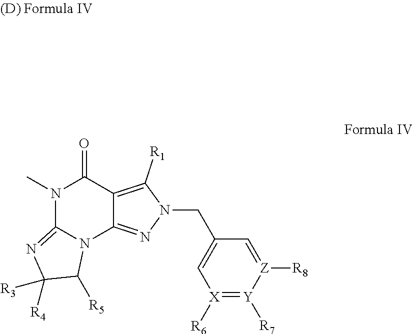
in free or salt form, wherein
(v) R₁ is C₁₋₄ alkyl (e.g., methyl or ethyl), or —NH(R₂), wherein R₂ is phenyl optionally substituted with halo (e.g., fluoro), for example, 4-fluorophenyl;
(vi) X, Y and Z are, independently, N or C;
(vii) R₃, R₄ and R₅ are independently H or C₁₋₄ alkyl (e.g., methyl); or R₃ is H and R₄ and R₅ together form a tri-methylene bridge (pref. wherein the R₄ and R₅ together have the cis configuration, e.g., where the carbons carrying R₄ and R₅ have the R and S configurations, respectively),
(viii) R₆, R₇ and R₈ are independently:
H,
pyrid-2-yl substituted with hydroxy, or
—S(O)₂—NH₂;
provided that when X, Y and/or Z are N, then R₆, R₇ and/or R₈, respectively, are not present; and
when X, Y and Z are all C, then at least one of R₆, R₇ or R₈ is —S(O)₂—NH₂ or pyrid-2-yl substituted with hydroxy,
in free or salt form; 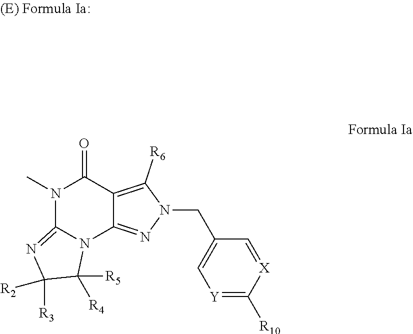
wherein
(iv) R₂ and R₅ are independently H or hydroxy and R₃ and R₄ together form a tri- or tetra-methylene bridge [pref. with the carbons carrying R₃ and R₄ having the R and S configuration respectively]; or R₂ and R₃ are each methyl and R₄ and R₅ are each H; or R₂, R₄ and R₅ are H and R₃ is isopropyl [pref. the carbon carrying R₃ having the R configuration];
(v) R₆ is (optionally halo-substituted) phenylamino, (optionally halo-substituted) benzylamino, C₁₋₄alkyl, or C₁₋₄alkyl sulfide; for example, phenylamino or 4-fluorophenylamino;
(vi) R₁₀ is C₁₋₄alkyl, methylcarbonyl, hydroxyethyl, carboxylic acid, sulfonamide, (optionally halo- or hydroxy-substituted) phenyl, (optionally halo- or hydroxy-substituted) pyridyl (for example 6-fluoropyrid-2-yl), or thiadiazolyl (e.g., 1,2,3-thiadiazol-4-yl); and
X and Y are independently C or N,
in free, pharmaceutically acceptable salt or prodrug form, including its enantiomers, diastereoisomers and racemates; 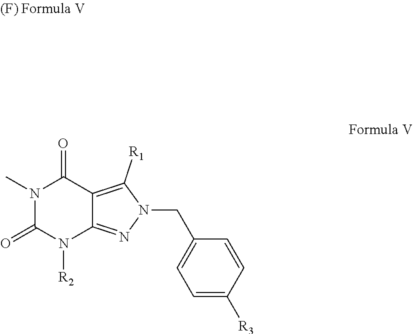
wherein
(iv) R₁ is —NH(R₄), wherein R₄ is phenyl optionally substituted with halo (e.g., fluoro), for example, 4-fluorophenyl;
(v) R₂ is H or C₁₋₆ alkyl (e.g., methyl, isobutyl or neopentyl);
(vi) R₃ is —SO₂NH₂ or —COOH;
in free or salt form; and/or 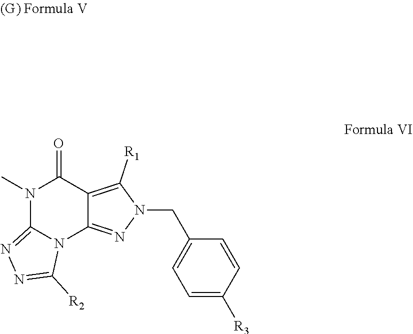
(iv) R₁ is —NH(R₄), wherein R₄ is phenyl optionally substituted with halo (e.g., fluoro), for example, 4-fluorophenyl;
(v) R₂ is H or C₁₋₆ alkyl (e.g., methyl or ethyl);
(vi) R₃ is H, halogen (e.g., bromo), C₁₋₆ alkyl (e.g., methyl), aryl optionally substituted with halogen (e.g., 4-fluorophenyl), heteroaryl optionally substituted with halogen (e.g., 6-fluoropyrid-2-yl or pyrid-2-yl), or acyl (e.g., acetyl),
in free or salt form.

12. The method according to claim 1, wherein the PDE1 inhibitor is selected from any of the following: 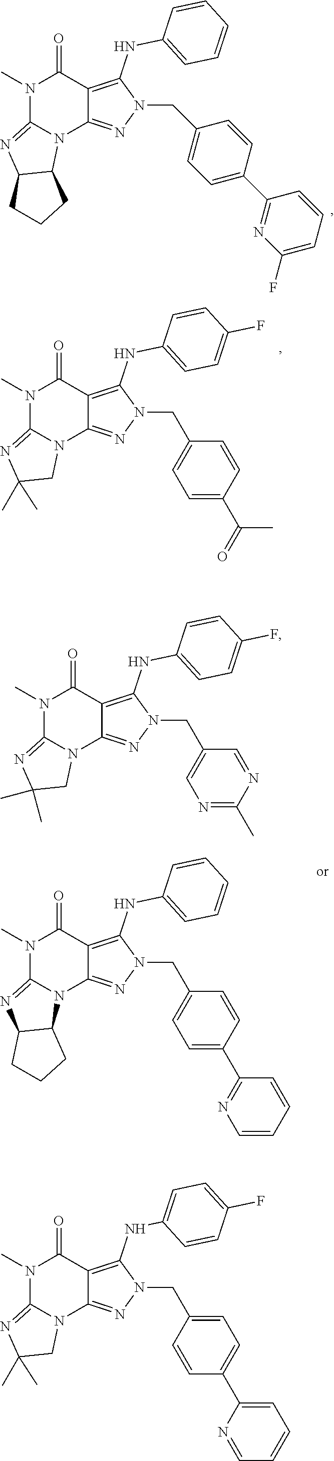
in free or pharmaceutically acceptable salt form.

13. The method according to claim 1, wherein the PDE1 inhibitor is administered in combination with a monoamine reuptake inhibitor (e.g., a dopamine reuptake inhibitor, a serotonin reuptake inhibitor, and/or an epinephrine reuptake inhibitor).

14. A pharmaceutical combination therapy comprising a pharmaceutically effective amount of a PDE1 inhibitor and a monoamine reuptake inhibitor (e.g., a dopamine reuptake inhibitor, a serotonin reuptake inhibitor, and/or an epinephrine reuptake inhibitor), for administration in a method according to claim 1.
